(19) United States
(12) Patent Application Publication
(10) Pub. No.: US 2025/0066828 A1
(43) Pub. Date: Feb. 27, 2025

(54) C-NUCLEOSIDE MONOPHOSPHATE SYNTHESIS

(71) Applicants: UAB BIOMATTER DESIGNS, Vilnius (LT); ENGINZYME AB, Solna (SE)

(72) Inventors: Martyna KOPLUNAITE, Vilnius (LT); Nina URBELIENE, Vilnius (LT); Rolandas MEŠKYS, Vilnius (LT); Diana IKASALAITE, Vilnius (LT); Linas ZAKRYS, Vilnius (LT); Laurynas KARPUS, Vilnius (LT); Federica RUGGIERI, Enskede (SE); Sascha GROBE, Solna (SE)

(21) Appl. No.: 18/724,381

(22) PCT Filed: Jan. 10, 2023
(86) PCT No.: PCT/EP2023/050489

§ 371 Date: Jun. 26, 2024

(30) Foreign Application Priority Data
Jan. 10, 2022 (GB) 2200248.9

(51) Int. Cl. C12P 19/38; C07H 19/10; C12N 9/10; C12N 9/16; C12N 9/88; C12N 9/90

(52) U.S. Cl. CPC C12P 19/385; C07H 19/10; C12N 9/1077; C12N 9/16; C12N 9/88; C12N 9/90; C12Y 204/02003; C12Y 301/03001; C12Y 402/0107; C12Y 504/02007

(57) ABSTRACT

Provided herein is a one-pot method of synthesising a C-nucleoside-5′-monophosphate such as pseudouridine-5′-monophosphate, from an N-nucleoside using a multi-step enzymatic pathway. The method comprises reacting the N-nucleoside with a phosphate source and nucleoside phosphorylase to form a pentose-1-phosphate intermediate, reacting the pentose-1-phosphate intermediate with a phosphomutase to form pentose-5-phosphate intermediate, and reacting the pentose-5-phosphate intermediate with a nucleoside-5′-phosphate C-glycosidase to form the C-nucleoside-5′-monophosphate.

### CROSS-REFERENCE TO RELATED APPLICATIONS

This application claims priority to United Kingdom patent application No. 2200248.9, filed Jan. 10, 2022, the disclosure of which is incorporated herein by reference in its entirety.

### FIELD OF THE INVENTION

The present invention generally relates to the synthesis of nucleoside derivatives. More specifically, the present invention relates to a method of synthesising C-nucleoside-5′-monophosphates from N-nucleosides using a multi-step enzymatic pathway. The method comprises reacting the N-nucleoside with a phosphate source and nucleoside phosphorylase to form a pentose-1-phosphate intermediate, reacting the pentose-1-phosphate intermediate with a phosphomutase to form pentose-5-phosphate intermediate, and reacting the pentose-5-phosphate intermediate with a nucleoside-5′-phosphate C-glycosidase to form the C-nucleoside-5′-monophosphate.

### BACKGROUND OF THE INVENTION

Nucleosides are the molecular building blocks of DNA and RNA. A nucleoside is a glycosylamine compound that comprises a pentose sugar linked to a nitrogenous base. In DNA, the pentose sugar is 2-deoxy-D-ribose, and in RNA, the pentose sugar is D-ribose.

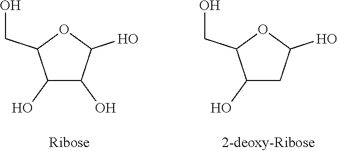 [SMILES: OCC1OC(O)C(O)C1O ; OCC1OC(O)CC1O]

Nitrogenous bases are heterocyclic organic compounds which may be categorised as pyrimidines or purines. Pyrimidine bases have the general structure of a six-membered ring comprising four carbon atoms and two nitrogen atoms. Purine bases comprise a pyrimidine ring fused with an imidazole ring. There are five primary or canonical bases as illustrated below: the purines adenine and guanine; and the pyrimidines cytosine, thymine, and uracil.

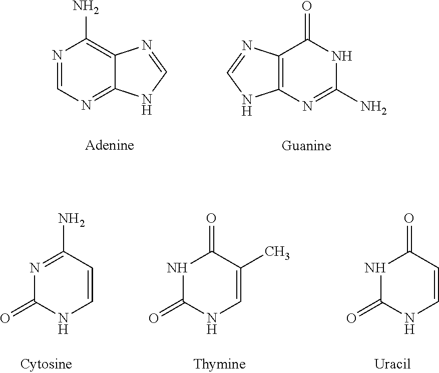

In living systems, DNA and RNA may also contain derivatives of canonical bases that have been modified after the formation of the nucleic acid. As an example, a common non-canonical base found in DNA is 5-methylcytosine. The methylation of cytosine plays a central role in the regulation of gene expression and consequent adaptation to environmental changes, differentiation, and development. As a further example, 7-methylguanine is often found at the 5′-end of mRNA (7-methyguanosine cap). This modification protects the mRNA from degradation and assists in translation of the mRNA. Altered modification patterns of nucleic acids have been observed in pathologies such as cancer. A vast number of synthetically modified bases or base analogues also exist, many of which serve as important biochemical tools.

Nucleosides, both synthetic and of natural product origin, have been widely used in clinical applications, particularly as anti-viral or anti-cancer agents. Nucleosides used in clinical applications are typically N-nucleosides in which the heterocyclic unit of the base is connected to the pentose moiety through an N-glycosidic (C—N) linkage. Notable examples include the anti-viral agent vidarabine and the anti-cancer agent, gemcitabine. However, N-nucleosides are susceptible to hydrolysis by N-glycosidases, limiting their therapeutic activity. This has raised interest in the development of C-nucleosides in which the canonical N-glycosidic bond between the base and the pentose sugar is replaced by a non-hydrolysable C-glycosidic (C—C) bond.

Notably, the synthetic C-nucleosides BCX4430 and GS6620 have been shown to be effective against filoviruses and the Hepatitis C virus, respectively. Originally developed for use against filoviruses, the synthetic C-nucleoside GS-5734 has now been evaluated against the SARS-CoV-2 virus. Other C-nucleosides, such as showdomycin, formycin, pseudouridimycin and minimycin, constitute an emerging class of natural microbial compounds that exhibit diverse biological activities, including antibiotic efficacy.

Pseudouridine is a C-nucleoside and an isomer of the N-nucleoside uridine. The structures of uridine and pseudouridine are provided for comparison below.

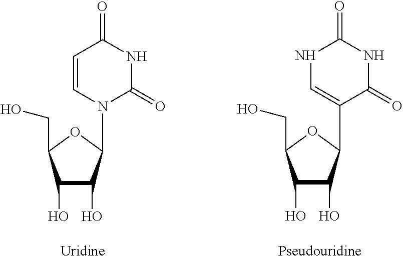

Pseudouridine is formed post-transcriptionally by pseudouridylate synthase (alternative term: pseudouridine-5′-monophophosphate glycosidase) in RNAs such as tRNA, ribosomal RNAs and small RNAs. The binding of uracil to ribose through C5 rather than through N1 allows the presence of an additional imino proton which can form a hydrogen bond with a water molecule. This in turn stabilises the secondary structure of RNA and reinforces base stacking interactions.

Pseudouridine and derivatives thereof have been used to replace uridine in mRNA-based vaccines in order to reduce the immunogenicity of the mRNA and increase antigen stability and expression. The Pfizer-BioNtech vaccine against SARS-CoV-2 (BNT162b2) and the Moderna Therapeutics vaccine against SARS-CoV-2 (mRNA-1273) both comprise mRNA in which uracil has been replaced with N1-methylpseudouridine. Quick adoption of mRNA-based vaccines and expansion of their application into different clinical areas creates a high commercial demand for pseudouridine and its derivatives.

Pseudouridine cannot readily be obtained by extracting from native RNAs in mammalian cell lines and tissues due to the relatively small proportion of pseudouridine. This is further reflected in the significantly higher retail price of pseudouridine or its derivatives as compared to uridine.

Attempts have been made to develop methods for synthesising pseudouridine and its derivatives. Once such method involves a multi-step semi-enzymatic synthesis of pseudouridine (Riley et al. 2021, Bioorg. Med. Chem. Lett. 44, 128105). In this method, schematically illustrated below, adenosine monophosphate (AMP) is subjected to depurination under acidic conditions and under a high temperature of ^{˜}100° C. to form ribose-5-phosphate. Ribose-5-phosphate is extracted and purified from the acidic mixture in which it is formed by isopropanol precipitation and anion exchange chromatography. The purified ribose-5-phosphate is then reacted with uracil in the presence of pseudouridine-5′-monophosphate glycosidase (ψMP glycosidase) to form pseudouridine-5′-monophosphate. Pseudouridine-5′-monophosphate is purified by column chromatography prior to reacting with bacterial alkaline phosphatase to generate pseudouridine. Purified pseudouridine is obtained by HPLC. However, the harsh conditions and multiple purification steps required in this method render the method time- and cost-inefficient.

Semi-Enzymatic Synthesis of Pseudouridine (Adapted from Riley et al. 2021)

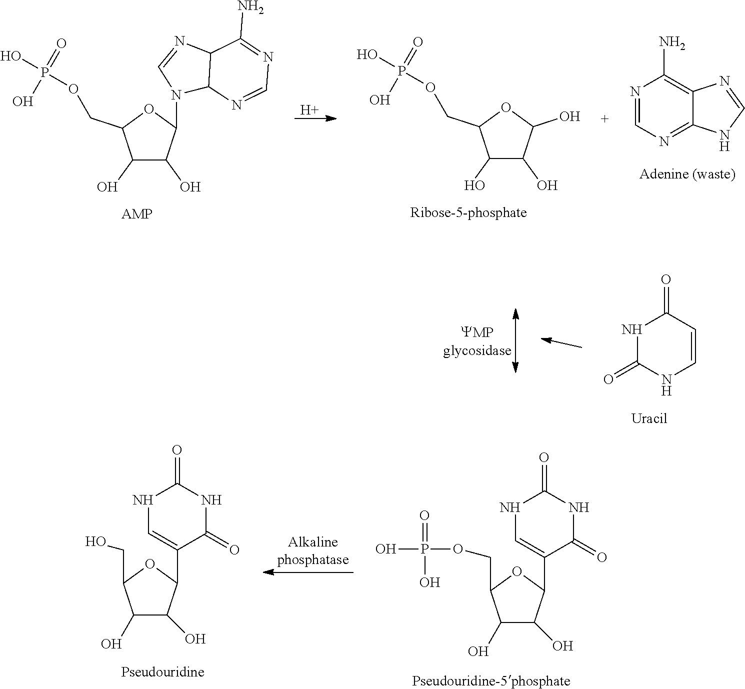

CN 114196715 discloses a similar method for the synthesis of pseudouridine. In this method, however, ribose-5-phosphate (obtained by hydrolysis of AMP) is reacted with uracil in the presence of a mutated pseudouridine-5′-monophosphate glycosidase to form pseudouridine-5′-monophosphate. The pseudouridine is then obtained following dephosphorylation and separation.

A further multi-step method for synthesising pseudouridine-5′-monophosphate is schematically illustrated below. In this method, ribose is reacted with a ribokinase in the presence of adenosine triphosphate (ATP) to form ribose-5-phosphate. Ribose-5-phosphate may then be converted to pseudouridine-5′-monophosphate and pseudouridine as in the above method by Riley et al. However, ATP is expensive and the method again requires multiple purification steps rendering it time- and cost-inefficient.

### Further Multi-Step Method for Synthesising Pseudouridine-5′-Monophosphate

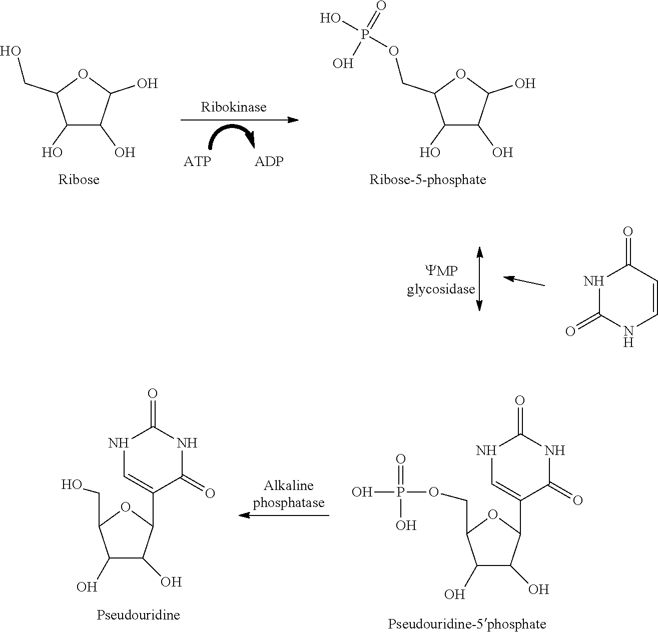 [SMILES: OCC1OC(O)C(O)C1O ; O=P(O)(O)OCC1OC(O)C(O)C1O ; O=c1cc[nH]c(=O)[nH]1 ; O=c1[nH]cc(C2OC(CO)C(O)C2O)c(=O)[nH]1 ; O=c1[nH]cc(C2OC(COP(=O)(O)O)C(O)C2O)c(=O)[nH]1]

It would therefore be desirable to provide more efficient methods of synthesising C-nucleosides such as pseudouridine and derivatives thereof, which overcome the disadvantages of existing methods.

### SUMMARY OF THE INVENTION

Accordingly, a method for synthesising a C-nucleoside 5′-monophosphate is provided, the method comprising:
a) reacting an N-nucleoside with a source of phosphate in the presence of a nucleoside phosphorylase to form a first intermediate, wherein the nucleoside comprises a pentose sugar and a first base, and wherein the first intermediate comprises a pentose-1-phosphate;
b) reacting the first intermediate comprising a pentose-1-phosphate with a phosphomutase to form a second intermediate, wherein the second intermediate comprises a pentose-5-phosphate;
c) reacting the second intermediate comprising a pentose-5-phosphate with a second base in the presence of a nucleoside-5′-phosphate C-glycosidase to form the C-nucleoside-5′-monophosphate.

Preferred features of the invention are defined in the dependent claims.

Advantageously, the cost of the starting materials in the method of the invention are low. Furthermore, the present inventors have unexpectedly found that the method of the invention can be performed as a one-pot reaction without the need for the separation or purification of any intermediate products. This further reduces costs and renders the method time-efficient.

### BRIEF DESCRIPTION OF THE FIGURES

Reference is made to the following description and drawings, in which:

FIG. 1 is a Thin Layer Chromatogram (TLC) for samples taken at various times from a reaction mixture comprising reactants for the synthesis of pseudouridine-5′-monophosphate.

FIG. 2 is an HPLC-MS chromatogram and peak table of a reaction mixture for the synthesis of pseudouridine-5′-monophosphate which has been treated with alkaline phosphatase.

FIG. 3 is an HPLC-MS chromatogram and peak table of commercially available pseudouridine.

FIG. 4 is an HPLC-MS chromatogram of a reaction mixture for the synthesis of pseudouridine-5′-monophosphate which has been treated with alkaline phosphatase representing protonated (top) and deprotonated (bottom) forms of pseudouridine.

FIG. 5A is an HPLC-MS chromatogram of a reaction mixture for the synthesis of pseudouridine-5′-monophosphate without alkaline phosphatase treatment illustrating uracil and pseudouridine-5′-monophosphate.

FIG. 5B is an HPLC-MS chromatogram of a reaction mixture for the synthesis of pseudouridine-5′-monophosphate without alkaline phosphatase treatment representing protonated (top) and deprotonated (bottom) forms of pseudouridine-5′-monophosphate.

FIG. 6 is a ¹H-NMR spectrum of synthesised pseudouridine-5′-monophosphate in D₂O.

FIG. 7 is a ¹³C-NMR spectrum of synthesised pseudouridine-5′-monophosphate in D₂O.

FIG. 8 is a ³¹P-NMR spectrum of synthesised pseudouridine-5′-monophosphate in D₂O.

FIG. 9 is a TLC of different reaction mixtures for synthesizing C-nucleoside 5′-monophosphates.

FIG. 10 shows the concentrations of pseudouridine monophosphate, uridine and uracil over time in a one-pot reaction on a 1 L scale.

FIG. 11 shows the uridine conversion and pseudouridine monophosphate yield in percent (%) after 24 h at different potassium phosphate buffer concentrations (in mM) at pH 8.

### DETAILED DESCRIPTION OF THE INVENTION

### Methods of Synthesising a C-Nucleoside 5′-Monophosphate

The present invention provides a method of synthesising a C-nucleoside 5′-monophosphate, the method comprising:
a) reacting an N-nucleoside with a source of phosphate in the presence of a nucleoside phosphorylase to form a first intermediate, wherein the nucleoside comprises a pentose sugar and a first base, and wherein the first intermediate comprises a pentose-1-phosphate;
b) reacting the first intermediate comprising a pentose-1-phosphate with a phosphomutase to form a second intermediate, wherein the second intermediate comprises a pentose-5-phosphate;
c) reacting the second intermediate comprising a pentose-5-phosphate with a second base in the presence of a nucleoside-5′-phosphate C-glycosidase to form the C-nucleoside 5′-monophosphate.

The term “comprising” or “comprises” as used herein denotes the inclusion of at least the features following the term, and does not exclude the inclusion of other features which have not been explicitly mentioned. The term may also denote an entity which consists of features following the term.

### N-Nucleoside

As discussed above, an N-nucleoside comprises a pentose sugar and a base which is typically nitrogenous, wherein the base is connected to the pentose moiety via an N-glycosidic (C—N) bond. Typically, in pyrimidine-based nucleosides, the glycosidic bond is between the C1′ and N−1, and in purine-based nucleosides, the glycosidic bond is between C1′ and N−9.

The pentose sugar is not limited but it is typically in a D-isomeric form. In preferred embodiments, the pentose comprises D-aldopentose. The D-aldopentose may be selected from D-ribose, 2-deoxy-D-ribose, D-arabinose, D-xylose, or D-lyxose and derivatives thereof. In preferred embodiments, the pentose comprises D-ribose. In other embodiments, the nucleoside may comprise a pentose as depicted in general Formula Ia, IIa, IIIa or IVa:

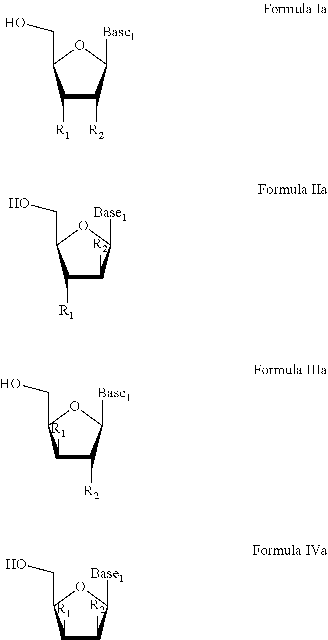

wherein R₁ and R₂ are independently selected from H, OH, OCH₃, F, NH₂, and N₃.

With specific reference to Formula Ia, the following substituents are provided: R₁ and R₂ are each OH; R₁ is OH and R₂ is H; R₁ and R₂ are H; R₁ is OH and R₂ is OCH₃; R₁ is N₃ and R₂ is H; R₁ is OH and R₂ is N₃; R₁ is H and R₂ is OH; R₁ is F and R₂ is OH; R₁ is OCH₃ and R₂ is OH; R₁ is OH and R₂ is F.

In a preferred embodiment, R₁ and R₂ are each OH (and the pentose comprises D-ribose). In another embodiment, R₁ is OH and R₂ is H (and the pentose comprises 2-deoxy-D-ribose). In yet another embodiment, R₁ is OH and R₂ is F (and the pentose comprises 2-deoxy-2-fluoro-D-ribose).

Whilst the above substituent selections have been provided with specific reference to Formula Ia, they are also relevant and applicable to Formulae IIa, IIIa, and IVa. In preferred embodiments, R₁ and R₂ are each OH such that the pentose of Formula Ia comprises D-arabinose, the pentose of Formula IIIa comprises D-xylose, and the pentose of Formula IVa comprises D-lyxose.

Whilst the furanose structures of pentose sugars have been depicted herein by Haworth projections, in the method of the invention, the pentose sugars may exist in other structural isomeric forms such as pyranose or linear forms, or a combination thereof.

Schematic representations of the reactions of the method according to the present invention, with N-nucleosides comprising a pentose according to Formula Ia, IIa, IIIa and IVa, respectively, are illustrated below. “A”, “B” and “C” refer to nucleoside phosphorylase, phosphomutase, and nucleoside-5′-monophosphate C-glycosidase, respectively.

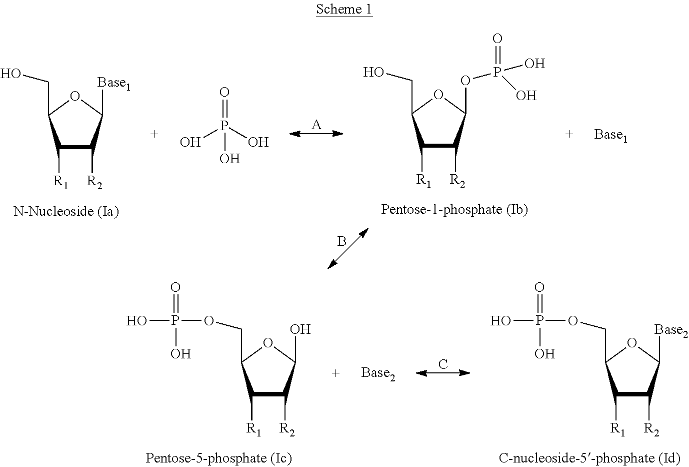

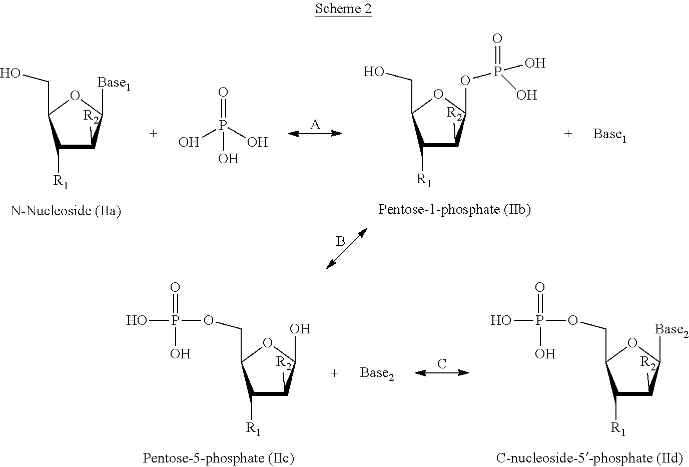

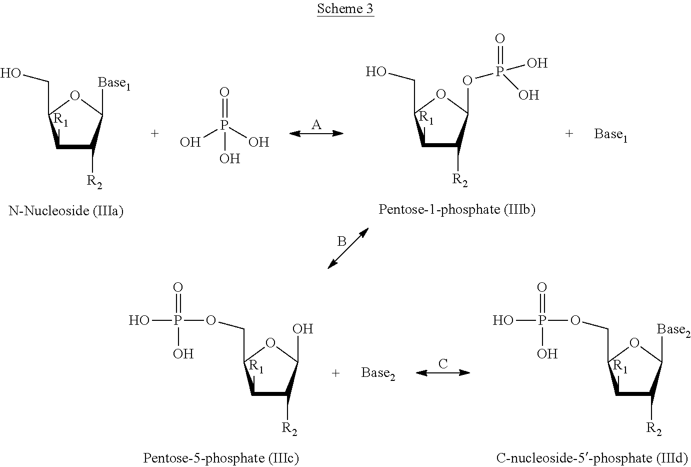

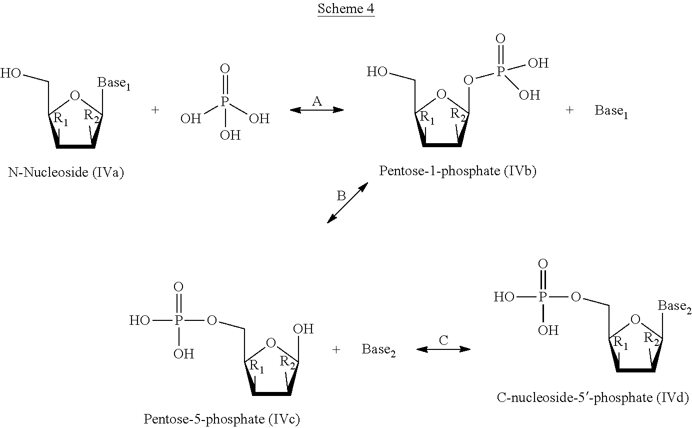

The first base of the N-nucleoside is not particularly limited and may comprise a standard purine or pyrimidine base selected from adenine, cytosine, guanine, thymine and uracil. Chemically modified bases are also envisaged within the methods of the present invention Chemically modified bases for use as the first base may include 3-methyl uracil, 6-aminouracil, 4-thiouracil, 2-thiouracil, hypoxanthine, xanthine, 7-methylguanine, 5, 6-dihydrouracil, 5-methylcytosine, and 5-(3-aminoallyl) uracil.

The first bases mentioned herein are illustrated below. For simplicity, the bases are illustrated within an N-nucleoside structure according to Formula Ia only. Analogous structures would be envisaged with Formulae IIa, IIIa and IVa, respectively.

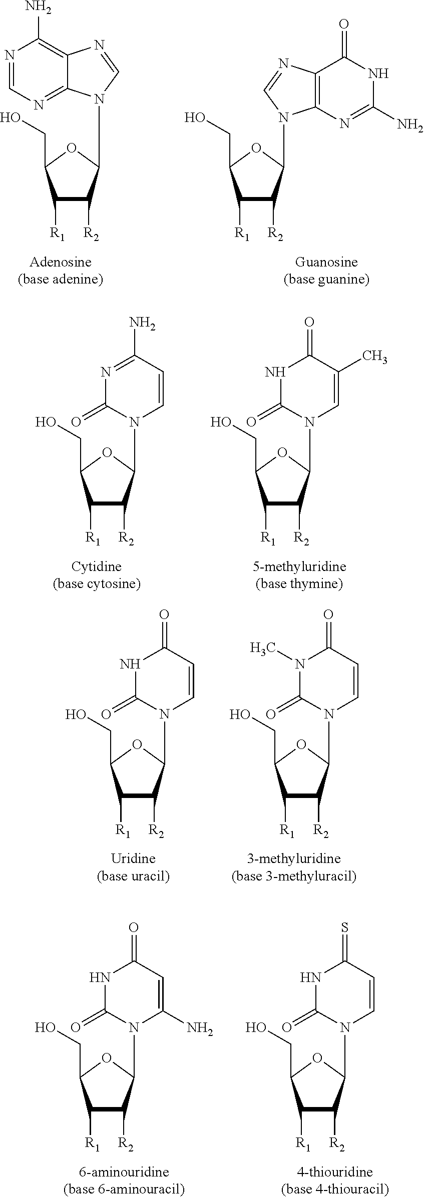

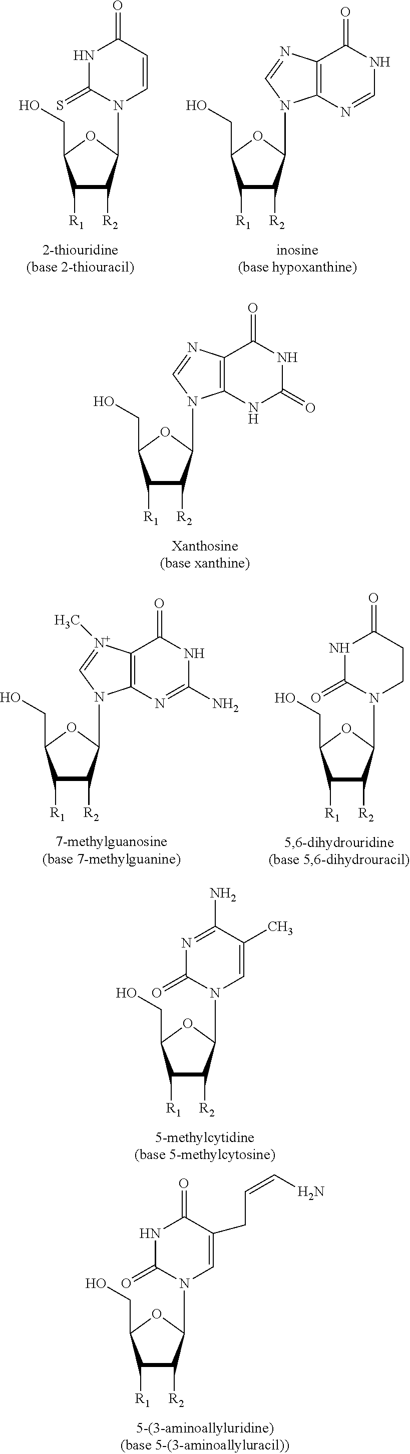

Other modified bases or base analogues which are capable of forming a N-glycosidic (C—N) bond with the pentose sugars disclosed herein may also be used in the reactions of the invention in some embodiments.

### One-Pot Reaction and Reaction Conditions

The method of the present invention may be advantageously performed as a “one-pot” reaction. By “one-pot” reaction it is meant that the synthesis of the C-nucleoside-5′-monophosphate is performed without any separation and purification of the intermediate compounds, preferably with all reactants provided in a single reactor. This advantageously saves time and resources whilst increasing chemical yield.

In preferred embodiments, the N-nucleoside, phosphate source, nucleoside phosphorylase, phosphomutase, second base and nucleoside-5′-phosphate C-glycosidase (reactants) are added simultaneously into a reactor. In other embodiments, one or more of the reactants are added sequentially into the reactor.

The reactions are preferably conducted at the optimum temperature and pH of the enzymes involved. Typically, the reactions are performed at about 37° C. or at the optimal temperature of the enzymes. However, the reactions may be performed at a temperature of from about 20° C. to about 37° C., or up to any temperature at which the enzymes retain activity. Typically, the reactions are performed at the optimal pH of the enzymes. For instance, the reactions may be performed at a pH of from about 6 to about 8 (such as from about 6 to about 7, or from about 7 to about 8) or at any pH at which the enzymes retain activity. In some embodiments, the reactions are performed at a pH of about 7. In some embodiments, the reactions are performed at a pH of about 8.

### Nucleoside Phosphorylase

Nucleoside phosphorylases suitable for use in the present invention are not particularly limited and include any enzyme capable of catalysing the reaction: N-Nucleoside↔D-pentose-1-phosphate+first base. Nucleoside phosphorylases generally have a wide specificity, and typically, any given nucleoside phosphorylase may act on several substrates. The nucleoside phosphorylase for use in the methods of the present invention may be selected based on the starting N-nucleoside, and in particular, based on the base moiety of the N-nucleoside. Nucleoside phosphorylases for use in the methods of the present invention are readily available commercially, and may also be obtained by established methods of recombinant expression.

In some embodiments, nucleoside phosphorylases that are employed include, but are not limited to: purine-nucleoside phosphorylase (EC 2.4.2.1), pyrimidine-nucleoside phosphorylase (EC 2.4.2.2), thymidine phosphorylase (EC 2.4.2.4): thymine, Uridine phosphorylase (EC 2.4.2.3) and Guanosine phosphorylase (EC 2.4.2.15). (The EC numbers provided herein refer to the Enzyme Commission numbers. This is an internationally accepted numerical classification scheme for enzymes. Enzyme Commission numbers are based on, and specify, the reactions catalysed by the enzymes, rather than the enzymes themselves).

In a preferred embodiment, the nucleoside phosphorylase comprises uridine phosphorylase. Other nucleoside phosphorylases having the same EC number as any of those listed above may also be used in the present invention.

In preferred embodiments, the N-nucleoside comprises guanine, adenine, hypoxanthine, xanthine or 7-methylguanine as the first base moiety, and the nucleoside phosphorylase that is used comprises a purine-nucleoside phosphorylase (EC 2.4.2.1). Such purine-nucleoside phosphorylases have e.g. been identified in Bacillus subtilis ((de Giuseppe et al. 2012, PLoS ONE vol. 7, e44282; Uniprot: 034925) and in Escherichia coli (Jensen et al. 1975, Eur. J. Biochem., vol. 51, pp. 253-265; Uniprot: P0ABP8).

In other preferred embodiments, the N-nucleoside comprises cytosine, thymine, 5,6-dihydrouracil, 5-methlylcytosine, 5-(3-aminoallyl) uracil, uracil, 3-methyluracil, 6-aminouracil, 4-thiouracil, or 2-thiouracil as the base moiety, and the nucleoside phosphorylase that is used comprises a pyrimidine-nucleoside phosphorylase (EC 2.4.2.2). Such pyrimidine-nucleoside phosphorylases have e.g. been identified in Bacillus stearothermophilus (Saunders et al. 1969, J. Biol. Chem., vol. 244, pp. 3691-3697; Pugmire et al. 1998, Structure, vol. 6, 1467-1479; Uniprot: P77836). Pyrimidine/pyrine nucleoside phosphylases have also been identified in Escherichia coli (Wen et al. 2022, Proteins vol. 90, pp. 1233-1241; Uniprot: P0C037) and in Acinetobacter baumannii (Vallenet et al. 2008, PLoS ONE vol. 3, e1805; Uniprot: BOVLV6).

In further preferred embodiments, the N-nucleoside comprises uracil as the first base moiety, and the nucleoside phosphorylase that is used comprises uridine phosphorylase (EC 2.4.2.3). Uridine phosphorylase have e.g. been identified in Klebsiella aerogenes (Veiko et al. 1998, Bioorg. Khim., vol. 24, pp. 381-387; UniProt ID: 008444) and Escherichia coli (Walton et al. 1989, Nucleic Acids Res. vol. 17, p. 6741; Uniprot: P12758).

In yet further embodiments, the N-nucleoside comprises thymine or uracil as the first base moiety, and the nucleoside phosphorylase that is used comprises thymidine phosphorylase (EC 2.4.2.4); or the N-nucleoside comprises guanine as the first base moiety, and the nucleoside phosphorylase that is used comprises guanosine phosphorylase (EC 2.4.2.4). Thymidine phosphorylase has been identified e.g. in Escherichia coli (Walter et al. 1990, J. Biol. Chem., vol. 265, pp. 14016-14022; Uniprot: P07650).

However, the above-mentioned combinations of phosporylases and base substrates are not exhaustive or limiting, and other combinations are envisaged and would be readily determined by the skilled person.

The source of phosphate in step a) of the methods of the present invention that is a required substrate of the nucleoside phosphorylase may comprise a compound selected from soluble alkaline or alkaline earth metal phosphates. In some embodiments, the source of phosphate comprises a compound selected from sodium dihydrogen phosphate, trisodium phosphate, sodium hydrogen phosphate, potassium dihydrogen phosphate, tripotassium phosphate, and potassium hydrogen phosphate. In a preferred embodiment, the source of phosphate comprises a potassium phosphate buffer of about pH 7. In another preferred embodiment, the source of phosphate comprises a potassium phosphate buffer of about pH 8.

In some embodiments, the concentration of nucleoside phosphorylase used in step a) is from about 0.02 to about 0.6 mg/mL, such as from about 0.3 to about 0.6 mg/mL, from about 0.4 to about 0.6 mg/ml, or about 0.5 mg/ml, or such as from about 0.02 to about 0.5 mg/mL, from about 0.02 to about 0.4 mg/mL, from about 0.02 to about 0.3 mg/mL, from about 0.02 to about 0.2 mg/mL, from about 0.02 to about 0.1 mg/mL, from about 0.02 to about 0.05 mg/mL, or from about 0.025 to about 0.035 mg/mL. In some embodiments, the concentration of nucleoside phosphorylase is about 0.02 mg/mL, about 0.025 mg/mL, about 0.03 mg/mL, about 0.035 mg/mL, about 0.04 mg/mL, about 0.045 mg/mL, or about 0.05 mg/mL. Concentrations of enzyme below 0.02 mg/mL may reduce the yield of the final C-nucleoside-5′-monophosphate, and concentrations of enzyme above 0.6 mg/mL may have limited economical utility.

### Phosphomutase

The phosphomutase used in step b) of the methods of the present invention is not particularly limited and includes any enzyme capable of catalysing the reaction: D-pentose-1-phosphate↔D-pentose-5-phosphate. Preferably, the phosphomutase is a phosphopentomutase. Phosphopentomutases also have wide specificity, and a given phosphopentomutase may act on a number of different D-pentose-1-phosphate intermediates to form the D-pentose-5-phosphate intermediates.

A preferred phosphopentomutase for use in the methods of the present invention comprises an enzyme with the EC number 5.4.2.7 such as deoB phosphopentomutase. deoB phophopentomutase may be isolated from several different bacterial strains including Escherichia coli, (Valentin-Hansen et al. 1984, Nucleic Acids Res., vol. 12, pp. 5211-5224; Uniprot: P0A6K6), Bacillus cereus (Panosian et al. 2011, J. Biol. Chem., vol. 268, pp. 8143-8054; Uniprot: Q818Z9), Streptococcus pneumoniae and Bacillus anthracis.

Other phosphopentomutases are commonly known and include, for example, phosphopentomutases isolated from Thermus thermophilus HB8 (GenBank ID: 3169853 or UniProt: Q5SLG9), Streptococcus thermophilus (GenBank ID: 66898918 or UniProt: Q5M482), and Saccharomyces cerevisiae S288C (GenBank ID: 855321 or UniProt: Q03262).

In preferred embodiments, step a) of the method of the invention comprises reacting an N-nucleoside comprising a D-ribose or 2-deoxy-D-ribose sugar according to Formula Ia, wherein R₁ and R₂ are each OH, or R₁ is OH and R₂ is H, respectively, with a phosphopentomutase (for example, deoB phosphopentose mutase (EC 5.4.2.7) or any other enzyme having the same enzyme classification as deoB phosphopentomutase), to form the first intermediate D-ribose-1-phosphate or 2-deoxy-D-ribose-1-phosphate.

In some embodiments, the concentration of phosphomutase used in step b) is from about 0.2 to about 0.6 mg/mL, such as from about 0.3 to about 0.6 mg/mL, from about 0.4 to about 0.6 mg/mL, or about 0.5 mg/mL, or such as from about 0.2 to about 0.5 mg/mL, from about 0.2 to about 0.4 mg/mL, or from about 0.2 to about 0.3 mg/mL. In some embodiments, the concentration of phosphomutase is about 0.2 mg/mL, about 0.25 mg/mL, about 0.3 mg/mL, or about 0.35 mg/mL. Concentrations of enzyme below 0.2 mg/mL may reduce the yield of the final C-nucleoside-5′-monophosphate, and concentrations of enzyme above 0.6 mg/mL may have limited economical utility.

Nucleoside-5′-monophosphate C-glycosidase

The nucleoside-5′-monophosphate C-glycosidase for use in the methods of the present invention is not particularly limited and includes any enzyme capable of catalysing the reaction: D-pentose-5-phosphate+second base↔C-nucleoside-5-′monophosphate. C-Nucleoside glycosidases generally have a wide specificity, and typically, a given C-Nucleoside glycosidase may act on several different substrates. C-Nucleoside glycosidases for use in the methods of the present invention may be obtained by established methods of recombinant expression.

A preferred C-Nucleoside glycosidase for use in the methods of the present invention is pseudouridylate synthase (alternatively termed pseudouridine-5′-monophopshate glycosidase) (EC 4.2.1.70) or any other enzyme having the same enzyme classification. Pseudouridylate synthase is a metabolic enzyme used in the nucleotide catabolic pathway in prokaryotes. Pseudouridylate synthase or related enzymes with comparable activity to pseudouridylate synthase have been identified and isolated from many prokaryotic sources. For example, YeiN (psuG) has been identified in Escherischia coli(Preumont et al. 2008, J. Biol. Chem., vol. 283, No. 37, pp. 25238-25246; Pfeiffer and Nidetzky 2020, Nat Commun. 2020 Dec. 8; 11(1):6270.), AInA has been identified in Streptomyces species (Oja et al. 2013, PNAS, vol. 100, no. 4, pp. 1291-1296), SdmA has been identified in Streptomyces showdoensi (Palmu et al. 2017, ACS Chemical Biology, vol. 12, pp. 1472-1477), and TmYeiN has been identified in Thermotoga maritima (Preumont et al. 2008, J. Biol. Chem., vol. 283, No. 37, pp. 25238-25246). Additionally, MinB (GenBank: QDX19370.1) has been identified in Streptomyces hygroscopicus (Kong et al. 2019, iScience, vol. 22, pp 430-440) and IndA (GenBank: AFV27435.1) has been identified in Streptomyces chromofuscus. Eukaryotic homologues of pseudouridylate synthase also exist as multifunctional enzymes. Any of the aforementioned enzymes would be suitable for the methods of the present invention.

In some embodiments, pseudouridylate synthase catalyses the 5-β-C-glycosylation of uracil and derivatives thereof from a pentose-5-phosphate substrate. The pentose may include any one of D-ribose (Formula Ic above wherein R₁ and R₂ are each OH), 2-deoxy-D-ribose (Formula Ic above wherein R₁ is OH and R₂ is H), 2-deoxy-2-fluoro-D-ribose (formula Ic above wherein R₁ is OH and R₂ is F); D-arabinose (Formula Iic above wherein R₁ and R₂ are each OH), D-xylose (Formula IIIc above wherein R₁ and R₂ are each OH) and D-lyxose (Formula Ivc above wherein R₁ and R₂ are each OH). Uracil derivatives may include, but are not limited to, 3-methyluracil, 6-aminouracil, 4-thiouracil, 2-thiouracil.

In some embodiments, step c) of the method of the present invention comprises reacting pseudouridylate synthase (for example, YeiN) with D-ribose-5-phosphate in the presence of a base selected from uracil, 3-methyluracil, 6-aminouracil, 4-thiouracil, or 2-thiouracil (the second base).

In some embodiments, step c) of the method of the present invention comprises reacting pseudouridylate synthase (for example, YeiN) with 2-deoxy-D-ribose-5-phosphate in the presence of a base selected from uracil, 3-methyluracil, 6-aminouracil, 4-thiouracil, or 2-thiouracil (the second base).

In some embodiments, step c) of the method of the present invention comprises reacting pseudouridylate synthase (for example, YeiN) with D-xylose-5-phosphate in the presence of a base selected from uracil, 3-methyluracil, 6-aminouracil, 4-thiouracil, or 2-thiouracil (the second base).

In some embodiments, step c) of the method of the present invention comprises reacting pseudouridylate synthase (for example, YeiN) with D-arabinose-5-phosphate in the presence of a base selected from uracil, 3-methyluracil, 6-aminouracil, 4-thiouracil, or 2-thiouracil (the second base).

In some embodiments, step c) of the method of the present invention comprises reacting pseudouridylate synthase (for example, YeiN) with D-lyxose-5-phosphate in the presence of a base selected from uracil, 3-methyluracil, 6-aminouracil, 4-thiouracil, or 2-thiouracil (the second base).

In some embodiments, step c) of the method of the present invention comprises reacting pseudouridylate synthase (for example, YeiN) with 2-deoxy-2-fluoro-D-ribose in the presence of a base selected from uracil, 3-methyluracil, 6-aminouracil, 4-thiouracil, or 2-thiouracil (the second base).

In the above embodiments, and with reference to Schemes 1 to 4 provided above, the pentose moiety of the pentose-5-phosphate that is the substrate of pseudouridylate synthase is derived from the N-nucleoside starting compound.

In embodiments where the first base and second base are different (see description of “wasteful” reaction below), the nucleoside-5′-monophosphate C-glycosidase is preferably reactive towards the second base but unreactive or less reactive towards the first base.

Without wishing to be bound by theory and with specific reference to uracil or a derivative thereof as a substrate, it is believed that the reaction catalysed by pseudouridylate synthase involves cleavage of the regular N1-C1′ glycosidic bond, 180° rotation of the uracil base along the N3-C6 axis, and coupling of C5 and C1′, in a reaction analogous to a Mannich addition, to form the C—C glycosidic bond. Analogous chemistries are expected to occur with other combinations of base and pentose substrates, and with other comparable C-nucleoside-5′-monophosphate glycosidase enzymes.

In some embodiments, the concentration of C-nucleoside-5′-monophosphate glycosidase used in step c) is from about 0.2 to about 0.6 mg/mL, such as from about 0.3 to about 0.6 mg/mL, from about 0.4 to about 0.6 mg/mL, or about 0.5 mg/mL, or such as from about 0.2 to about 0.5 mg/mL, from about 0.2 to about 0.4 mg/mL, or from about 0.25 to about 0.35 mg/mL. In some embodiments, the concentration of C-nucleoside-5′-monophosphate glycosidase is about 0.2 mg/mL, about 0.25 mg/mL, about 0.3 mg/mL, about 0.35 mg/mL, or about 0.4 mg/mL. Concentrations of enzyme below 0.2 mg/mL may reduce the yield of the final C-nucleoside-5′-monophosphate, and concentrations of enzyme above 0.6 mg/mL may have limited economical utility.

In some embodiments, the nucleoside phosphorylase, phosphomutase and/or nucleoside 5′-phosphate C-glycosidase may comprise thermophilic enzymes, for example, as found in thermophilic bacteria. Thermophilic enzymes may be advantageous due to their increased robustness and stability (for example, under high temperatures and in the presence of common protein denaturants) as compared to their non-thermophilic counterparts. These properties may facilitate storage of the enzymes, enable enzymatic reactions to be run for longer periods of time, and facilitate isolation of the enzymes from reaction mixtures by methods such as ultrafiltration for subsequent re-use. For example, the thermophilic enzyme may be obtained by recombinant methods comprising: over-expressing the enzyme in E. coli or other suitable host cells, lysing the cells, heat-treating the cell lysate to inactivate all native E. coli enzymes (the thermophilic enzyme would retain activity in these conditions), filtering/centrifuging the heat-treated lysate to remove insoluble particles, and using the crude enzyme preparation for the enzymatic synthesis methods of the present invention.

### Second Base

The second base is also not particularly limited and may include a standard or chemically modified base as defined herein. Additional bases that may act as the second base include 2-amino-1H-pyrrole-5-carboxylate and 1,4-Napthoquinone. Other modified bases or base analogues which are capable of forming a C-glycosidic bond with the pentose-5-phosphate compounds disclosed herein may also be used in the invention.

### “Wasteless” Reaction

In some embodiments, the first base and the second base are the same. In these reactions, the first base produced as a waste product in step a) is re-used to form the C-nucleoside-5′-monophosphate in step c). Accordingly, there is no “waste” of base.

In these embodiments, the first and second base are preferably selected from uracil, 3-methyluracil, 6-aminouracil, 4-thiouracil and 2-thiouracil.

In a specific embodiment, the following method of synthesising pseudouridine-5′-monophosphate is provided, the method comprising:
a) reacting uridine with a source of phosphate in the presence of a nucleoside phosphorylase to form ribose-1-phosphate as a first intermediate and uracil;
b) reacting pentose-1-phosphate with a phosphomutase to form ribose-5-phosphate as a second intermediate;
c) reacting ribose-5-phosphate with uracil produced in step a) in the presence of a nucleoside-5′-phosphate C-glycosidase to form pseudouridine-5′-monophosphate.

The method is illustrated schematically below (Scheme 5). “A”, “B” and “C” refer to nucleoside phosphorylase, phosphomutase, and nucleoside-5′-phosphate C-glycosidase, respectively.

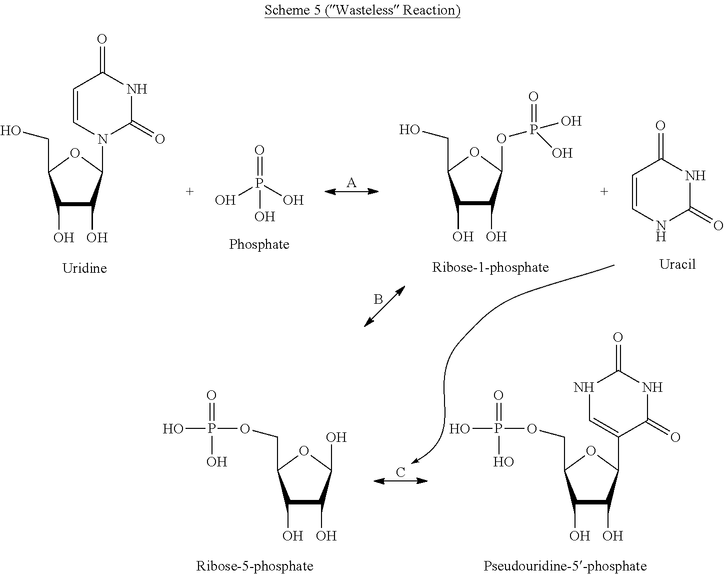

In the above “wasteless” method, the nucleoside phosphorylase, phosphomutase, and nucleoside 5′-phosphate C-glycosidase may be as described above. Preferably, the nucleoside phosphorylase comprise uridine phosphorylase, the phosphomutase comprises phosphopentomutase DeoB, and/or the nucleoside-5′-phosphate C-glycosidase comprises pseudouridylate synthase (psuG or yeiN).

In another embodiment of the “wasteless” reaction, and with further reference to Scheme 5, 2-thiouridine, 4-thiouridine, or 4-thio-2′-deoxyuridine is used in place of uridine as the starting nucleoside to form 2-thio-pseudouridine-5-phosphate, 4-thio-pseudouridine-5′phosphate, and 4-thio-2′-deoxy-pseudouridine-5′-phosphate, respectively. The nucleoside phosphorylase, phosphomutase, and nucleoside 5′-phosphate C-glycosidase used in the reactions may be as described above. Preferably, the nucleoside phosphorylase comprises uridine phosphorylase, the phosphomutase comprises phosphopentomutase DeoB, and/or the nucleoside-5′-phosphate C-glycosidase comprises pseudouridylate synthase (psuG or yeiN).

### “Wasteful” Reaction

In some embodiments, the first base is different to the second base. In these embodiments, the first base may be selected from cytosine, guanine, thymine, adenine, hypoxanthine, xanthine, 7-methylguanine, 5, 6-dihydrouracil, 5-methylcytosine, and 5-(3-aminoallyl) uracil. The second base may be selected from uracil, 3-methyluracil, 6-aminouracil, 4-thiouracil, 2-thiouracil, 2-amino-1H-pyrrole-5-carboxylate, and 1,4-napthoquinone. In these embodiments, it is desirable that the nucleoside-5′-phosphate C-glycosidase is reactive towards the second base but unreactive or less active towards the first base, to ensure that the reaction proceeds in favour of formation of an N-nucleoside-5′-monophosphate.

In a specific embodiment, the following method of synthesising N-3 methylpseudouridine-5′-monophosphate is provided, the method comprising:
a) reacting 5-methyluridine with a source of phosphate in the presence of a nucleoside phosphorylase to form ribose-1-phosphate as a first intermediate and thymine as a waste product;
b) reacting ribose-1-phosphate with a phosphomutase to form ribose-5-phosphate as a second intermediate;
c) reacting ribose-5-phosphate with N3-methyluracil in the presence of a nucleoside-5′-phosphate C-glycosidase to form N-3 methylpseudouridine-5′-monophosphate.

The method is illustrated schematically below (Scheme 6). “A”, “B” and “C” refer to nucleoside phosphorylase, phosphomutase, and nucleoside-5′-phosphate C-glycosidase, respectively.

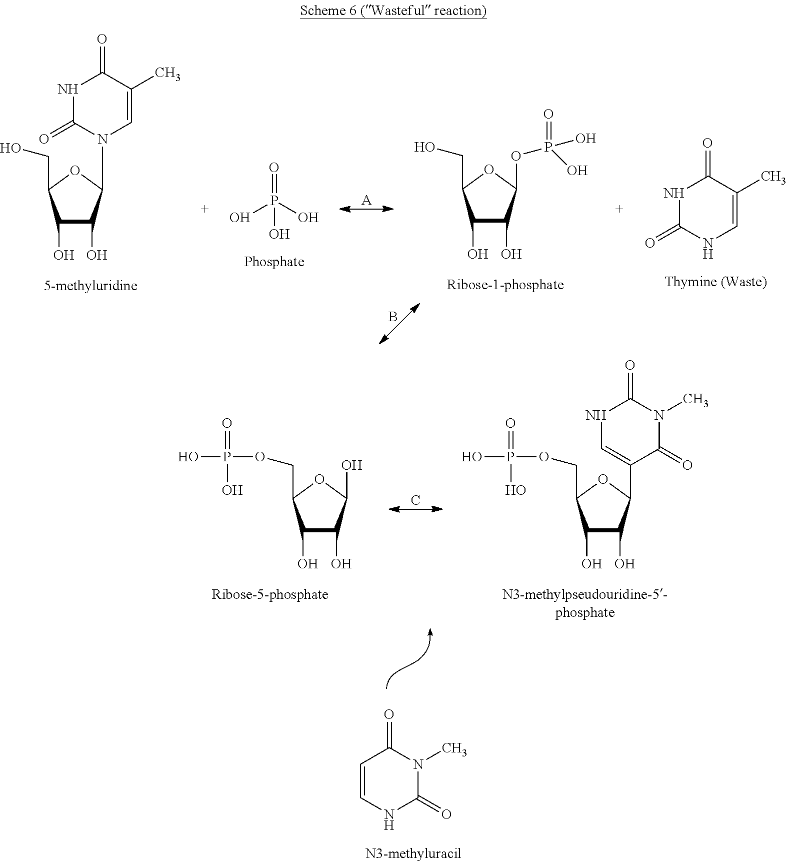

In the above “wasteful” method, the nucleoside phosphorylase, phosphomutase, and nucleoside 5′-phosphate C-glycosidase may be as described above. Preferably, the nucleoside phosphorylase comprises uridine phosphorylase or thymidine phosphorylase, the phosphomutase comprises phosphopentomutase DeoB, and/or the nucleoside 5′-phosphate C-glycosidase comprises pseudouridine-5′-phosphate glycosidase (for example, psuG/Yei N).

Purification of C-nucleoside-5′-monophosphate

The C-nucleoside-5′-monophosphate produced by the methods of the present invention may be subject to further separation and/or purification steps. In some embodiments, the C-nucleoside 5′ monophosphate is separated from the reaction mixture by one or more methods selected from High Performance Liquid Chromatography (HPLC), ion-exchange chromatography and reverse phase chromatography. In some embodiments, the C-nucleoside-5′-monophosphate is separated from the reaction mixture and/or purified by crystallization.

Depending on the desired application, the C-nucleoside-5′-monophosphate produced by the methods described herein may be treated with a phosphatase enzyme, optionally prior to any separation or purification steps, to generate a C-nucleoside (and phosphate waste product). As such, the present invention further provides a method of producing a C-nucleoside. In some embodiments, the phosphatase enzyme is an alkaline phosphatase enzyme. In some embodiments, the phosphatase enzyme is a HAD-like phosphatase, more specifically a HAD hydrolase subfamily 1A, e.g. Uniprot ID Q181K6 from Clostridioides difficile. The phosphatase is commercially available or readily produced by standard recombinant methods.

The C-nucleoside produced by the methods of the present invention may be subject to further separation and/or purification steps. In some embodiments, the C-nucleoside is separated from the reaction mixture by one or more methods selected from High Performance Liquid Chromatography (HPLC), ion-exchange chromatography and reverse phase chromatography. In some embodiments, the C-nucleoside is separated from the reaction mixture and/or purified by crystallization.

### Immobilization of Enzymes on Solid Support

When enzymes are used in organic synthesis (such as in organic solvents), they often tend to aggregate, precipitate and/or unfold (i.e., denature). In some embodiments, therefore, the enzymes used in the method disclosed herein are immobilized on a solid support and used as catalysts in an immobilized state. Such immobilization may improve the stability of the enzymes and allow for reaction conditions which the enzymes normally would not tolerate. The use of enzymes in an immobilized state also facilitates the separation from the reaction mixture and allows for recovery of the catalyst. Additionally, immobilization makes it possible to use immobilized enzymes in much higher concentrations than would have been possible with the free enzymes in solution. Methods for the immobilization of enzymes on a solid support are known in the art.

In some embodiments, the solid support is a rigid porous material having a pore diameter of from about 20 nm to about 100 nm, such as from about 20 to about 60 nm. In some embodiments, the solid support displays limited swelling in solvents and is chemically and dimensionally stable in most organic media and aqueous environments at pH below 10. In some embodiments, the solid support material is controlled porosity glass (CPG) or hybrid controlled porosity glass (hybrid CPG), as disclosed in WO 2015/115993.

In some embodiments, the enzymes comprise a metal affinity tag such as a polyhistidine tag (i.e., a His-tag of 2-8 consecutive histidine residues, preferably 6 consecutive histidine residues), to facilitate immobilization of the enzyme via a chelated metal on the solid support.

In some embodiments, each of the three enzymes used in the method disclosed herein (i.e., the nucleoside phosphorylase, the phosphomutase and the nucleoside-5′-monophosphate C-glycosidase) is individually immobilized on a solid support. In some embodiments, two or three of the enzymes are co-immobilized on the same solid support.

In some embodiments, the immobilized enzymes are used in batch or continuous stirred tank reactors. In some embodiments, the immobilized enzymes are packed within a fixed-bed reactor suitable for operation in a continuous-flow mode, such as a continuous flow fixed bed reactor.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The invention will now be described by the following examples which do not limit the invention in any respect. All cited documents and references mentioned herein are incorporated by reference in their entireties.

### EXPERIMENTAL METHODS

### Analytical Methods for Examples 3-11

For the analysis and separation of the compounds uridine, uracil, pseudouridine, and pseudouridine monophosphate the following setup was used: Instruments: Thermo scientific Vanquish, Agilent 1260 Infinity II, column: HILICON iHILIC-(P) Classic column (length×ID 100 mm×2.1), mobile phases: A: acetonitrile, B: 100 mM ammonium formate pH 5.8, gradient: 0 min-15% B, 2 min-15% B, 2.5 min-40% B, 5.5 min-40% B, 5.5 min-switch back to 15% B, hold for 5 minutes, flowrate: 0.3 mL/min, injection volume 2 μL. Sample preparation for analytics of uridine, uracil, pseudouridine, and pseudouridine monophosphate: 10 μL reaction sample was combined with 50 μL of an internal standard stock solution (10 mM caffeine), followed by the addition of 940 μL HPLC-grade water. The solution was vortexed for 15 seconds and then 500 μL of the resulting solution was mixed with 500 μL of acetonitrile (ACN) followed by vortexing for a further 15 seconds. The solution had a 200× dilution and a final caffeine concentration of 0.25 mM (standard) and was used for HPLC analysis. If caffeine was present an internal standard in the reaction mixture the HPLC sample preparation was as follows: 10 μL reaction sample was combined with 990 μL HPLC-grade water. The solution was mixed by vortexing for 15 seconds and then 500 μL of the resulting mixture was added to 500 μL of ACN followed by 15 seconds vortexing. The solution had a 200× dilution and a final caffeine concentration of 0.25 mM (standard) and was used for HPLC analysis.

For the analysis of phosphate, ribose-1-phosphate, ribose-5-phosphate the following setup was used: Instrument: Dionex Intergion HPIC, Column: Dionex IonPac AS11-HC-4 um RFIC & HPIC, column temperature 35° C., flow rate: 0.38 mL/min, detector: conductivity detector, detector temperature 35° C., injection volume 10 μL, eluent: KOH in water, gradient: 0 min-10 mM KOH, 6 min-35 mM KOH, 7 min-80 mM KOH, 9 min-80 mM KOH, 9 min 10 mM KOH, 13 min-10 mM KOH. Sample preparation for the analysis of the ribose-phosphates: 10 μL reaction sample was combined with 200 μL ACN LC-MS grade followed by the addition of 100 μL 10 mM ammonium formate in micropure water. 690 μL micropure water was then added and the solution was vortexed for 15 seconds. 500 μL of the resulting solution was mixed with 500 μL of micropure water and vortexed again for 15 seconds. The solution had a 200× dilution and a final ammonium formate concentration of 0.5 mM (as standard) and was used for HPLC analysis.

### Examples

### Example 1

### Production of Pseudouridine Monophosphate

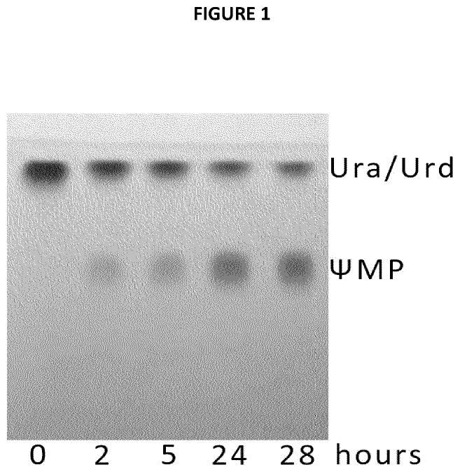

A reaction mixture containing 0.48 mg/mL uridine phosphorylase, 0.4 mg/mL phosphopentomutase, 0.5 mg/mL pseudouridine-5′-monophosphate (ψMP) glycosidase, 70 mM uridine, 0.5 mM MnCl₂ and 125 mM potassium phosphate buffer (pH 7.0) in a total volume of 25 mL was shaken at 37° C. The reaction was monitored by Thin Layer Chromatography (TLC) for 28 hours. As can be seen from FIG. 1, the amount of uracil/uridine decreased over time as the amount of ψMP increased, confirming effective ψMP synthesis.

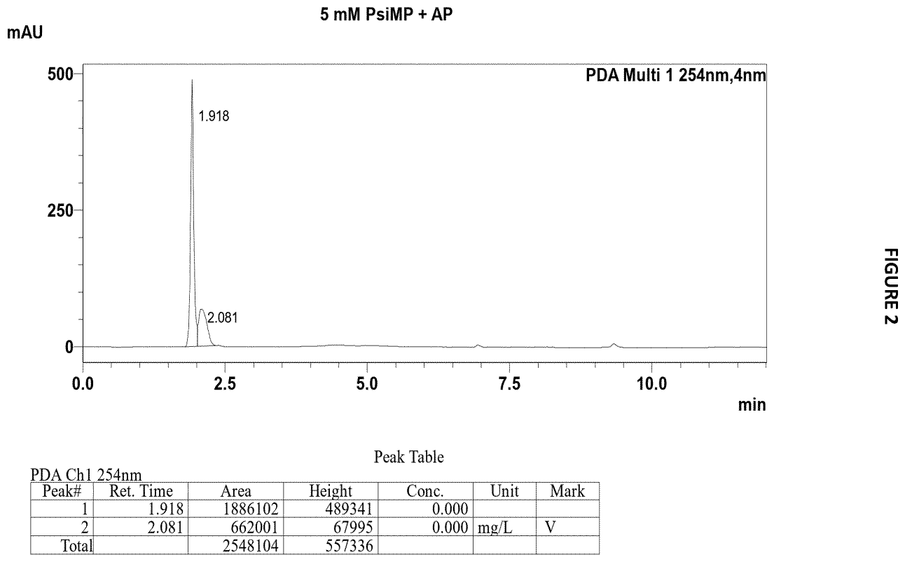

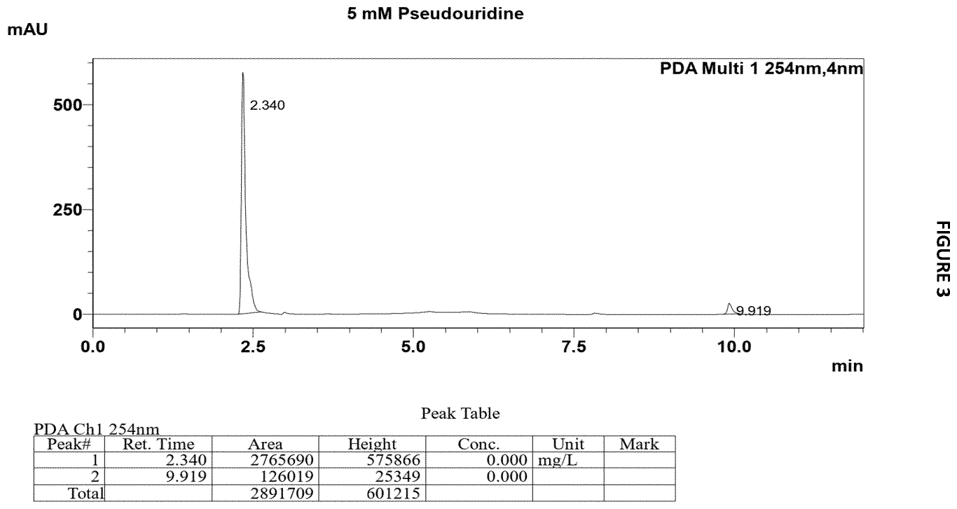

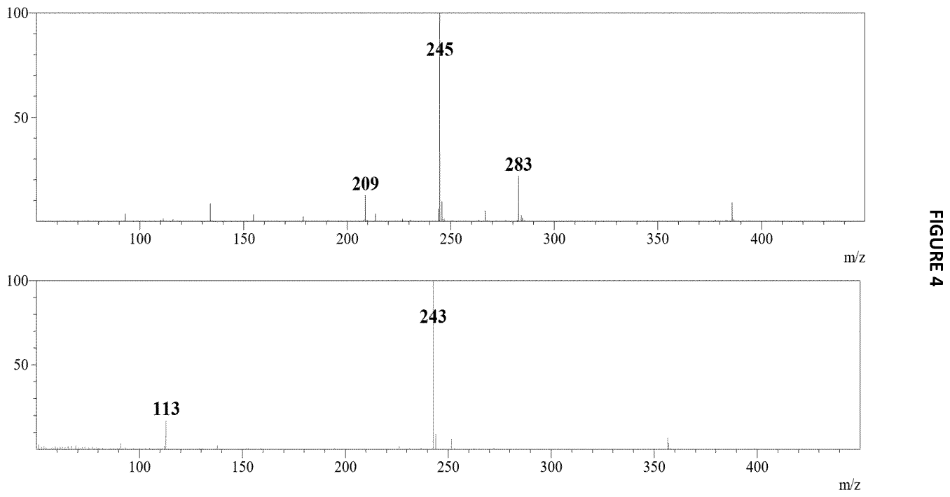

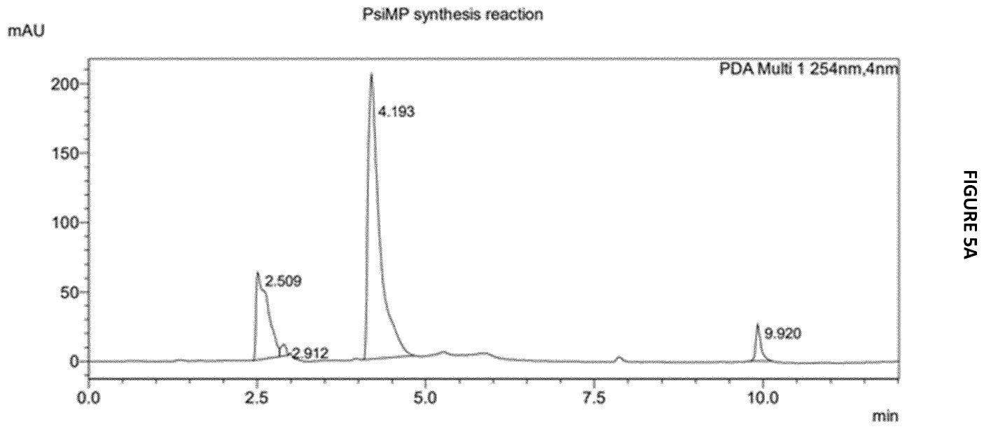

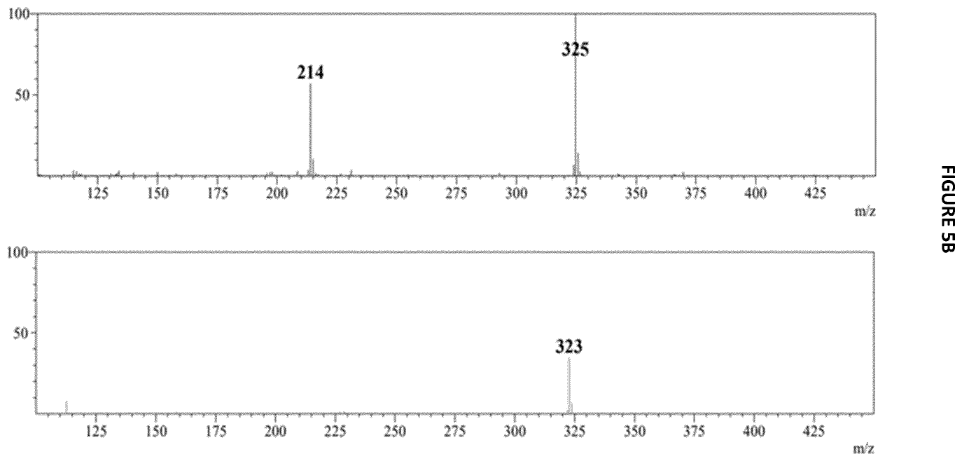

The identity of synthesised ψMP was confirmed by HPLC-MS and NMR spectroscopy. For HPLC-MS analysis, samples taken from the reaction mixture were treated with alkaline phosphatase to remove phosphate. FIG. 2 is an HPLC-MS chromatogram for the alkaline phosphatase-treated reaction mixture in which two peaks were observed at 254 nm. The first peak at an elution time of 1.918 minutes represents pseudouridine. The second peak at an elution time of 2.081 minutes represents uracil. FIG. 3 is a comparative HPLC-MS chromatogram for commercially available pseudouridine. An elution peak corresponding to pseudouridine is observed at a slightly longer retention time of 2.34 seconds due to the presence of organic solvents (methanol and acetonitrile) used for sample preparation. FIG. 4 is an HPLC-MS mass spectrum of material in peak with retention time of 1.918 minutes from FIG. 2. It demonstrates that detected ions are equal in mass to protonated pseudouridine with MS (ESI+): m/z 244.80 [M+H]+(top) and deprotonated pseudouridine with MS (ESI+): m/z 242.75 [M−H]⁻(bottom) (The molecular weight of pseudouridine is 244 g/mol). FIG. 5A is an HPLC-MS chromatogram for the reaction mixture prior to treatment with alkaline phosphatase. Unconsumed uracil is eluted at 2.509 minutes and ψMP is eluted at 4.193 minutes. FIG. 5B is an HPLC-MS mass spectrum for ψMP peak in FIG. 5A, showing that detected ions are equal in mass to protonated ψMP with MS (ESI+): m/z 324.75 [M⁺H]⁺ (top) and deprotonated ψMP with MS (ESI+): m/z 322.75 [M⁻H]⁻(bottom). (The molecular weight of ψMP is 324.18 g/mol.).

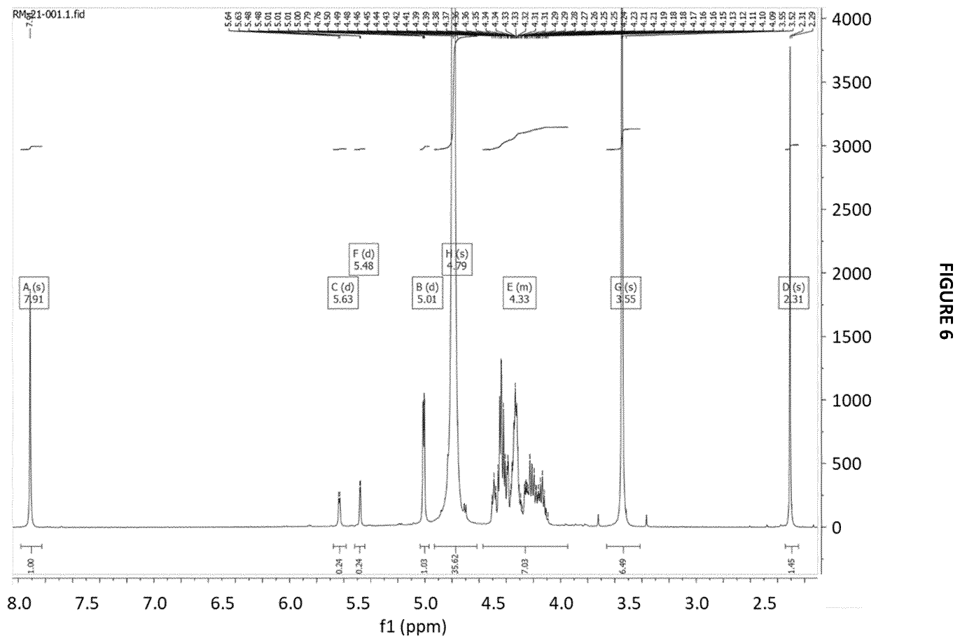

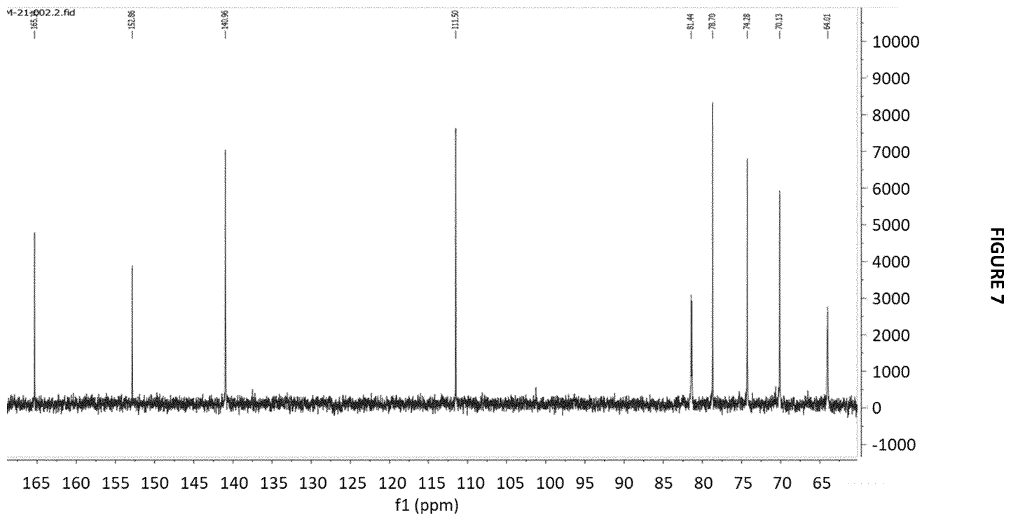

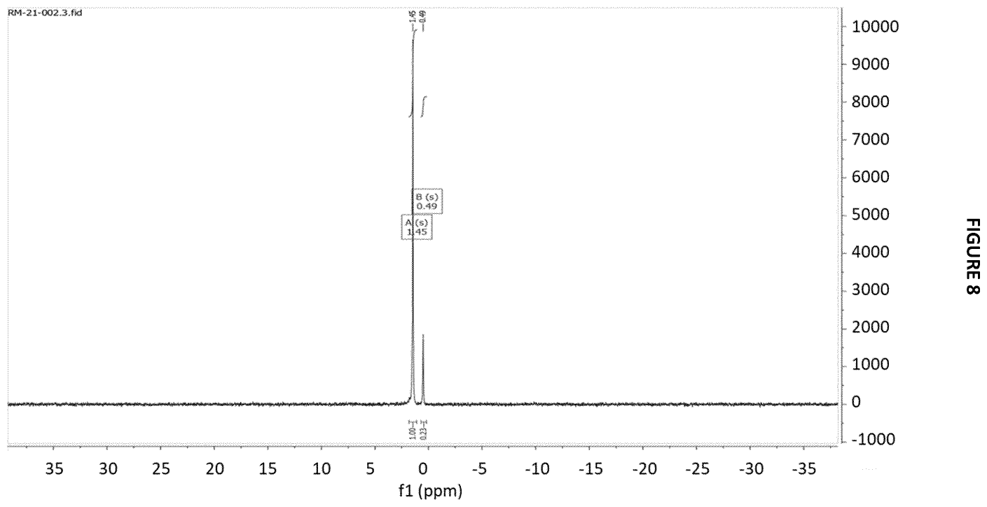

For NMR spectroscopy, synthesised ψMP was purified from the reaction mixture by ion-exchange chromatography (solid phase DEAE Sephadex A-25, eluent: 0.01-0.1 M NaCl) followed by reverse phase chromatography (C18 30 g. column, eluent: water). ¹H-NMR, ¹³C-NMR and 31^{P}-NMR spectra were recorded for the purified material to further confirm the identity of ψMP. FIG. 6 represents the ¹H-NMR spectrum of synthesised ψMP in D₂O. ψMP is characterized by two specific signals: a doublet at 4.80 ppm (1′-C hydrogen) and a singlet at 7.77 ppm (6-C hydrogen). Small signals at 5.24 ppm and 5.39 ppm represent ribose-1-phosphate or ribose-5-phosphate. FIG. 7 represents the ¹³C-NMR spectrum of synthesised ψMP in D₂O. The nine different signals represent the nine different carbon atoms of ψMP. FIG. 8 represents the ³¹P-NMR spectrum of synthesised ψMP in D₂O The major signal at 1.45 ppm represents the phosphorus atom of synthesised pseudouridine-5′-monophosphate and the smaller signal at 0.49 ppm is from ribose-1′-phosphate or ribose-5′-phosphate.

In order to measure reaction yield, samples of the reaction mixture were treated with alkaline phosphatase and analysed by HPLC-MS. The amount of pseudouridine in the sample was calculated by comparing the area of the pseudouridine peak in the measured sample (1886102 area units) to the area of pseudouridine peak in a standard sample (2765690 area units) with a known concentrations as provided in FIG. 3. Approximately 380 mg of ψMP formed in a 25 mL reaction which gives a yield of ^{˜}70%.

### Example 2

### Production of C-nucleoside-5′-monophosphates from Different N-nucleosides

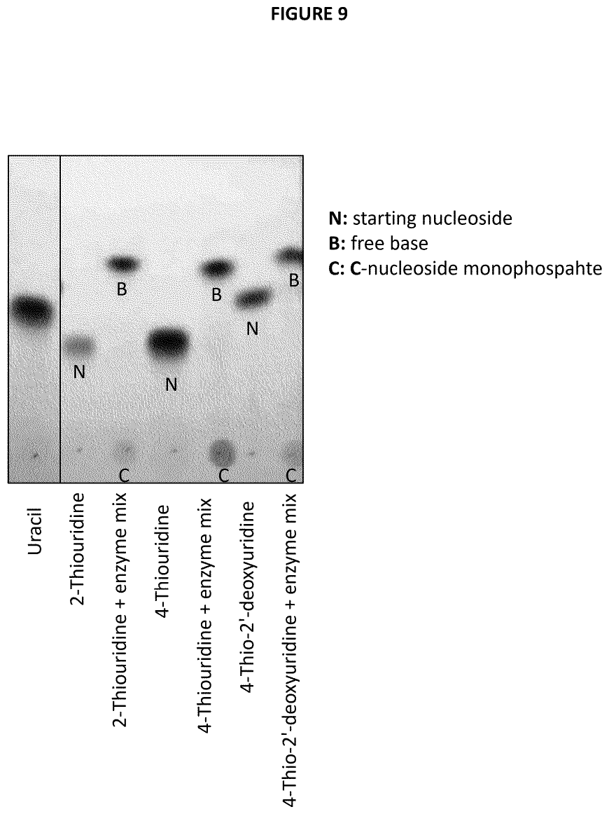

A reaction mixture containing 0.48 mg/mL uridine phosphorylase, 0.4 mg/mL phosphopentomutase, 0.5 mg/mL pseudouridine-5′-monophosphate (ψMP) glycosidase, 0.5 mM MnCl₂ and 125 mM potassium phosphate buffer (pH 7.0) was mixed with 10 mM 2-Thiouridine or 10 mM 4-Thiouridine or 10 mM 4-Thio-2′-deoxyuridine in a total volume of 0.1 mL was shaken at 37° C. for 16 hours. Resulting products were analysed with TLC as demonstrated by FIG. 9. C-nucleoside-5′-monophosphate formation is observed from all three starting nucleosides.

### Example 3

### Production of Pseudouridine Monophosphate on a 1-Liter Scale

A reaction mixture containing 0.03 mg/mL uridine phosphorylase, 0.26 mg/mL phosphopentomutase, 0.29 mg/mL pseudouridine-5′-monophosphate (ψMP) glycosidase, 200 mM uridine, 0.5 mM MnCl₂ and 240 mM potassium phosphate buffer pH 8.0 in a total volume of 1 L was stirred at 300 rpm and 37° C. in a jacketed 1 L stirred tank reactor (Radleys, batch reactor, glass). The reaction was monitored by the high-performance liquid chromatography (HPLC) method described, over the time course of 24.5 h. Measurements were performed in duplicate and the average values were determined.

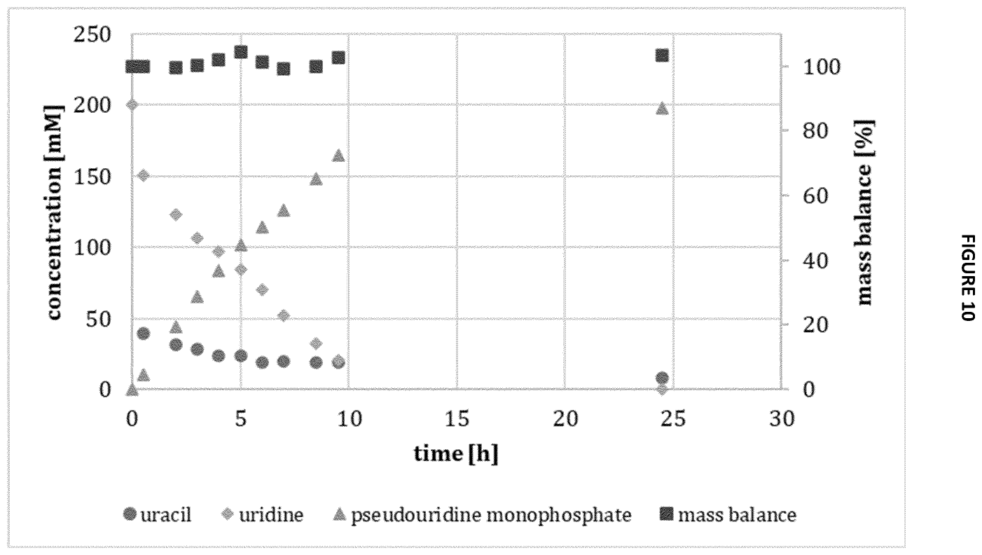

As shown in FIG. 10, pseudouridine monophosphate concentration constantly increased while the uridine concentration decreased. The level of uracil steadily decreased over time while higher pseudouridine monophosphate formation was reached. After 24.5 h the following product distribution was reached: 0.264 mM uridine, 8.147 mM uracil, 191.58 mM pseudouridine monophosphate. The ribose-phosphate intermediates were analyzed using HPLC ion chromatography revealing 1.0 mM of ribose-1-phosphate, and 6.2 mM of ribose-5-phosphate after 24.5 h. Product, substrate, and intermediates were identified based on HPLC analysis in comparison to commercially available standards (Sigma Aldrich, Merck, BOCSCI Inc.).

After 24.5 h, a uridine conversion of 99.7% was reached together with a pseudouridine monophosphate yield of 95.8% correlating to approximately 62 g of pseudouridine monophosphate, thereby demonstrating an effective scalable enzyme cascade reaction.

### Example 4

### Conversion of Pseudouridine Monophosphate to Pseudouridine

The material obtained from Example 3 was filtered (Filtering system: Amicon® Stirred Cell, filters: Ultrafiltration Disk, 10 kDa NMW, 76 mm diameter regenerated cellulose) and used directly as the reaction mixture for the dephosphorylation step. 5 mM MgCl₂ and 0.12 g/L phosphatase (Uniprot: Q181K6, origin: Clostridioides difficile (strain 630), stored in 50 mM Tris pH 8.0 with a concentration of 2.38 mg/mL) were added to the reaction mixture which was stirred at 300 rpm and 37° C. in a jacketed 1 L stirred tank reactor (Radleys, batch reactor, glass). The reaction was monitored by HPLC over 23 h.

After 23 h the following product distribution was obtained: 0.11 mM uridine, 4.77 mM uracil, 184 mM pseudouridine, 2 mM ribose-5-phosphate, 1.9 mM pseudouridine monophosphate. The reaction mixture was filtered (Filtering system: Amicon® Stirred Cell, filters: Ultrafiltration Disk, 10 kDa NMW, 76 mm diameter regenerated cellulose) and frozen. The example demonstrates an efficient dephosphorylation step working on a liter scale with a conversion of pseudouridine monophosphate of 98.7% and a pseudouridine yield of 96.2% which corresponds to 44.9 g/L.

### Example 5

### Purification and Crystallization of Pseudouridine from the Crude Reaction Mixture

An 8 mL aliquot of the material obtained from Example 4 was evaporated to dryness in vacuo using a rotary evaporator followed by further azeotropic drying with i-PrOH (1 mL). The resulting solids were redissolved in 2 mL of hot (60° C.) distilled water affording a total volume ^{˜}2.25 mL. A 1.8 mL aliquot of this mixture was cooled to RT whereupon small, white crystals appeared. The mixture was further cooled to 4° C. and allowed to sit overnight at this temperature. The resulting white crystals were filtered and washed with cold ethanol (0.5 mL), followed by drying. 104 mg of white, crystalline pseudouridine was obtained (Yield 38%). The purity of the crystals was determined by HPLC as described. Results: pseudouridine>99%, uracil 0.3%, pseudouridine monophosphate ^{˜}0.1%, uridine<0.1%.

### Example 6

### Conversion of Uridine to Pseudouridine-5′-monophosphate at Increased Uridine Concentration (200 mM) in the Presence of Different Potassium Phosphate Buffer Concentrations

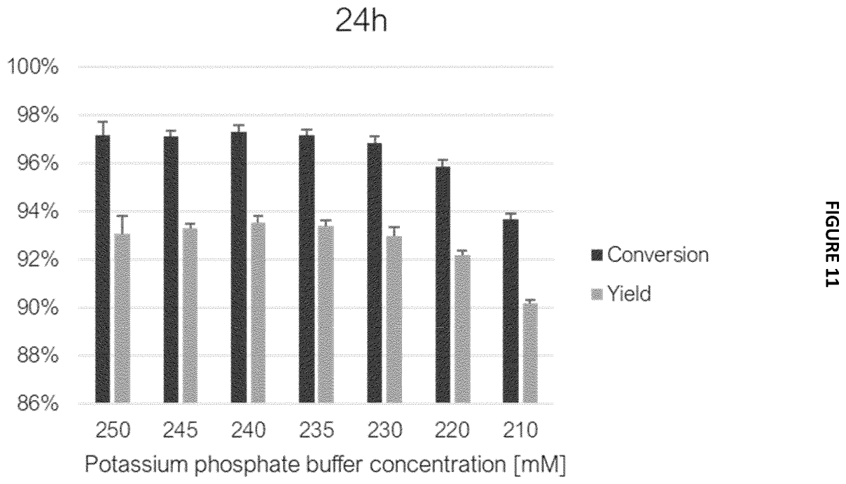

A set of 1 mL reaction mixtures was prepared in a 96-deep well plate (2 mL, round well) containing 0.14 mg/mL phosphorylase, 0.26 mg/mL phosphopentomutase, 0.29 mg/mL pseudouridine-5′-monophosphate (ψMP) glycosidase, 200 mM uridine, 0.5 mM MnCl₂, 50 mM caffeine (as an internal standard for the analytics), and variable concentrations of potassium phosphate buffer at pH 8.0 (250, 245, 240, 235, 230, 220, 210 mM). The reaction mixtures were incubated at 37° C. and 800 rpm in a plate shaker (MB100-4A Thermoshaker, Allsheng) for 24 h. Measurements were performed in triplicate (n=3). A decrease in uridine conversion and pseudouridine monophosphate yield was observed when the potassium phosphate buffer (pH 8.0) concentration is below 235 mM (see FIG. 11; the average of the measurements being displayed with indicated error bars). With a potassium phosphate buffer concentration of 240 mM at pH 8.0 the following product distribution was obtained: 187 mM pseudouridine monophosphate, 5 mM uridine, and 8 mM uracil. This corresponds to a uridine conversion of 97.5% and a pseudouridine monophosphate yield of 93.5%. Thus, an increase in substrate load is possible if the buffer capacity is increased in a similar manner.

### Example 7

### Conversion of Uridine to Pseudouridine-5′-monophosphate in the Presence of a Reduced Enzyme Concentration

50 mL of a reaction mixture containing 0.03 mg/mL phosphorylase, 0.26 mg/mL phosphopentomutase, 0.29 mg/mL pseudouridine-5′-monophosphate (ψMP) glycosidase, 240 mM potassium phosphate buffer pH 8.0, 0.5 mM MnCl₂ was prepared in an Erlenmeyer shake flask. The reaction mixture was incubated at 37° C. and 150 rpm (25 mm orbital shaker, New Brunswick Innova 42) for 24 h. After 24 h the following product composition was obtained: 0.3 mM uridine, 4.2 mM uracil, 0.4 mM pseudouridine, 195.2 mM pseudouridine monophosphate. This procedure resulted in a uridine conversion of 99.9% and a pseudouridine monophosphate yield of 97.6%. Furthermore, the phosphate and ribose-phosphate contents were determined to be 0.2 mM ribose-1-phosphate, 2.1 mM ribose-5-phosphate, and 30.8 mM of phosphate. Thus, a high uridine conversion and a pseudouridine monophosphate yield can be achieved with an enzyme concentration below 0.3 mg/mL per enzyme.

### Example 8

### Conversion of Uridine to Pseudouridine-5′-monophosphate in Potassium Phosphate Buffers with Different pH

A set of 1 mL reaction mixtures was prepared in a 96-deep well plate (2 mL, round wells) containing 0.14 mg/mL phosphorylase, 0.26 mg/mL phosphopentomutase, 0.29 mg/mL pseudouridine-5′-monophosphate (ψMP) glycosidase, 70 mM uridine, 0.5 mM MnCl₂, 50 mM caffeine (internal standard) with 200 mM potassium phosphate at different pHs (6, 7, 8). The reaction mixtures were incubated at 37° C. and 800 rpm in a plate shaker (MB100-4A Thermoshaker, Allsheng) for 24 h. After 24 h for the reactions running at pH 6 the product composition was: 21.82 mM uridine, 24.96 mM uracil, 23.21 mM pseudouridine monophosphate. This corresponds to a uridine conversion of 68.8% and a pseudouridine monophosphate yield of 33.1%. The reaction running at pH 7 after 24 h reaction time contained 2.0 mM uridine, 11.74 mM uracil, and 56.24 mM pseudouridine monophosphate corresponding to a uridine conversion of 97.1% and pseudouridine monophosphate yield of 79.9%. The reaction running at pH 8 after 24 h reaction time contained 0.25 mM uridine, 6.32 mM uracil, and 63.42 mM pseudouridine monophosphate, corresponding to a uridine conversion of 99.6% and a pseudouridine monophosphate yield of 90.9%. The results demonstrate that a reaction at pH 8 in potassium phosphate buffer is favorable compared to pH 6 or pH 7.

### Example 9

### Effect of Variation of Enzyme Concentrations on Conversion of Uridine to Pseudouridine-5′-monophosphate

A set of 1 mL reaction mixtures was prepared in a 96-deep well plate (2 mL, round wells) containing 50 mM potassium phosphate buffer pH 8.0, 200 mM uridine, 0.5 mM MnCl₂, 50 mM caffeine (internal standard) and the concentrations of the phosphorylase (udp), the phosphopentomutase (deoB) and the pseudouridine-5′-monophosphate (ψMP) glycosidase (psuG) were varied for one, two or all three enzymes by ±10% or ±15%. As shown below, this resulted in an evaluated concentration range for the phosphorylase of from 0.0255 to 0.0345 mg/mL, for the phosphopentomutase of from 0.221 to 0.299 mg/mL, and for the pseudouridine-5′-monophosphate (ψMP) glycosidase of from 0.2465 to 0.3335 mg/mL:

| | Enzyme concentration (mg/mL) | | | | |
|---|---|---|---|---|---|
| Enzyme | — | +10% | −10% | +15% | −15% |
| udp | 0.03 | 0.033 | 0.027 | 0.0345 | 0.0255 |
| deoB | 0.26 | 0.286 | 0.234 | 0.299 | 0.221 |
| psuG | 0.29 | 0.319 | 0.261 | 0.3335 | 0.2465 |

The product formation was monitored over 24 h. It was observed that the tested enzyme ratios had the same reaction profile and analyte distribution over the reaction course of 24 h. Even with all three enzymes decreased by 15%, the reaction appeared to proceed at the same rate. The results thus demonstrate a robust cascade reaction that tolerates deviations of enzyme amounts of ±15%.

### Example 10

### The Appropriate Enzyme Ratio Enables Pseudouridine-5′-monophosphate Formation

A 1 mL reaction mixture was prepared in a 96-deep well plate (round wells) containing 0.03 mg/mL phosphorylase, 0.26 mg/mL phosphopentomutase, 0.29 mg/mL pseudouridine-5′-monophosphate (ψMP) glycosidase, 70 mM uridine, 0.5 mM MnCl₂, 50 mM caffeine (internal standard for the analytics) and 200 mM potassium phosphate buffer pH 7.0. The mixture was incubated at 37° C. for 24 h. After 24 h the following product formation was observed: 51 mM pseudouridine monophosphate, 15.4 mM uracil, 3.6 mM uridine. This corresponds to a pseudouridine monophosphate yield of 72% and a uridine conversion of 95%. The yield is comparable with the yield of example 1 (^{˜}70%), but in the present example lower individual and overall enzyme concentrations were used. The results demonstrate that the enzyme load alone is not the only factor to consider for reaching high yields of pseudouridine-5′-monophosphate. Together with example 9, the results demonstrate that the enzyme ratio of phosphorylase, phosphopentomutase, pseudouridine-5′-monophosphate (ψMP) glycosidase is also important.

### Example 11

### Dephosphorylation of Pseudouridine-5′-monophosphate

A reaction mixture was prepared containing 5.1 mM uracil, 0.17 mM uridine, 50 mM caffeine (internal standard), 184.1 mM pseudouridine monophosphate, 5 mM MgCl₂ and 0.09 mg/mL phosphatase (Uniprot: Q181K6, origin: Clostridioides difficile (strain 630), stored in 50 mM Tris pH 8.0 with a concentration of 2.07 mg/mL). The resulting 785 mL reaction mixture was stirred for 23.5 h at 300 rpm and 37° C. in a jacketed 1 L stirred tank reactor (Radleys, batch reactor, glass). The reaction was monitored by HPLC over 23.5 h.

After 23.5 h the following product distribution was obtained: 0.15 mM uridine, 5.6 mM uracil, 181.7 mM pseudouridine, and 2 mM pseudouridine monophosphate. The reaction mixture was filtered (Filtering system: Amicon® Stirred Cell, filters: Ultrafiltration Disk, 10 kDa NMW, 76 mm diameter regenerated cellulose) and frozen.

1. A method of synthesising a C-nucleoside-5′-monophosphate, the method comprising:
a) reacting an N-nucleoside with a source of phosphate in the presence of a nucleoside phosphorylase to form a first intermediate, wherein the nucleoside comprises a pentose sugar and a first base, and wherein the first intermediate comprises a pentose-1-phosphate;
b) reacting the first intermediate comprising a pentose-1-phosphate with a phosphomutase to form a second intermediate, wherein the second intermediate comprises a pentose-5-phosphate; and
c) reacting the second intermediate comprising a pentose-5-phosphate with a second base in the presence of a nucleoside-5′-monophosphate C-glycosidase to form the C-nucleoside-5′-monophosphate.

2. The method of claim 1, wherein steps a), b) and c) are performed as a one-pot reaction.

3. The method of claim 1, wherein the N-nucleoside comprises a compound according to Formula Ia; the first intermediate comprises a compound according to Formula Ib; the second intermediate comprises a compound according to Formula Ic; and the C-nucleoside-5′-monophosphate comprises a compound according to Formula Id; 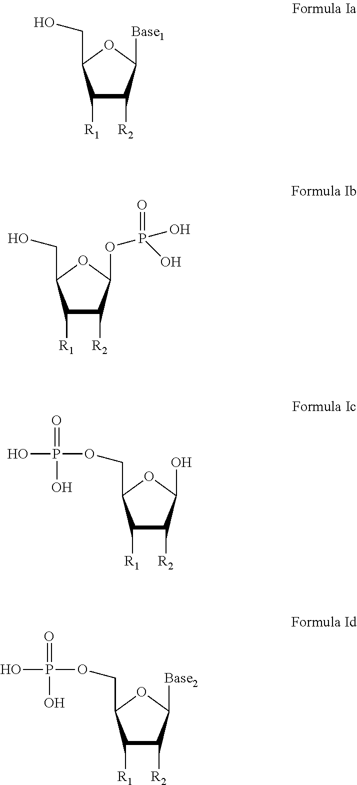
wherein R₁ and R₂ are independently selected from H, OH, OCH₃, F, NH₂, and N₃.

4. The method of claim 3, wherein R₁ and R₂ are each OH.

5. The method of claim 3, wherein R₁ is OH and R₂ is H, or R₁ and R₂ are H, or R₁ is OH and R₂ is OCH₃, or R₁ is N₃ and R₂ is H, or R₁ is OH and R₂ is N₃, or R₁ is H and R₂ is OH, or R₁ is F and R₂ is OH, or R₁ is OCH₃ and R₂ is OH, or R₁ is OH and R₂ is F.

6. The method of claim 1, wherein the first base is selected from uracil, 3-methyluracil, 6-aminouracil, 4-thiouracil, and 2-thiouracil.

7. The method of claim 1, wherein the second base is selected from uracil, 3-methyluracil, 6-aminouracil, 4-thiouracil, and 2-thiouracil.

8. The method of claim 1, wherein the first base is the same as the second base.

9. The method of claim 8, wherein the second base of step c) is the first base produced as a by-product of the reaction of step a).

10. The method of claim 8, wherein the first and second base comprise uracil.

11. The method of claim 1, wherein the N-nucleoside in step a) comprises uridine and the C-nucleoside-5′-monophosphate comprises pseudouridine-5′-monophosphate.

12. The method of claim 8, wherein the first and second base comprise 4-thiouracil or 2-thiouracil.

13. The method of claim 1, wherein the first base and second base are different.

14. The method of claim 13, wherein the nucleoside-5′phosphate C-glycosidase is reactive towards the second base but is unreactive towards the first base.

15. The method of claim 13, wherein the first base is selected from cytosine, guanine, thymine, adenine, hypoxanthine, xanthine, 7-methylguanine, 5, 6-dihydrouracil, 5-methylcytosine, and 5-(3-aminoallyl) uracil and/or the second base is selected from uracil, 3-methyluracil, 6-aminouracil, 4-thiouracil, 2-thiouracil, 2-amino-1H-pyrrole-5-carboxylate, and 1,4-napthoquinone.

16. The method of claim 1, wherein the first base comprises thymine, the second base comprises N3-methyluracil, the N-nucleoside in step a) comprises 5-methyluridine, and the C-nucleoside-5′-monophosphate comprises N3-methylpseudouridine-5′-monophosphate.

17. The method of claim 1, wherein the N-nucleoside comprises a compound according to Formula IIa, the first intermediate comprises a compound according to Formula IIb, the second intermediate comprises a compound according to Formula IIc, and the C-nucleoside 5′ monophosphate comprises a compound according to Formula IId; 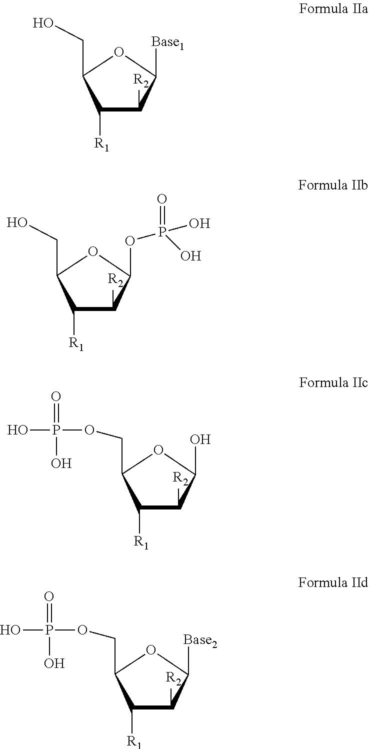
wherein R₁ and R₂ are independently selected from H, OH, OCH₃, F, NH₂, and N₃.

18. The method of claim 17, wherein R₁ and R₂ are each OH.

19. The method of claim 1, wherein in step a), the N-nucleoside comprises a compound according to Formula IIIa, the first intermediate comprises a compound according to Formula IIIb, the second intermediate comprises a compound according to Formula IIIc, and the C-nucleoside 5′ monophosphate comprises a compound according to Formula IIId; 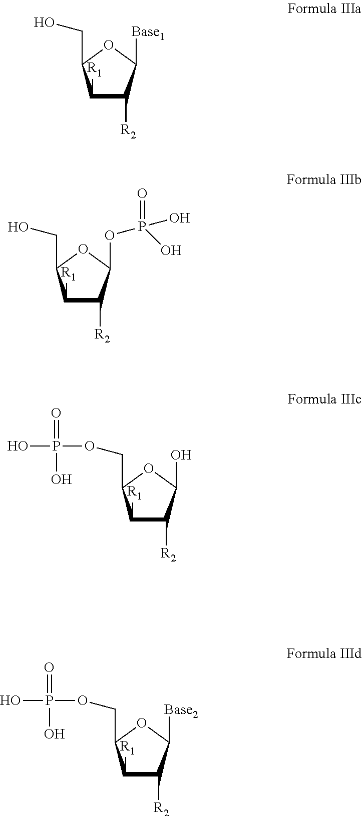
wherein R₁ and R₂ are independently selected from H, OH, OCH₃, F, NH₂, and N₃.

20. The method of claim 19, wherein R₁ and R₂ are each OH.

21. The method of claim 1, wherein the N-nucleoside comprises a compound according to Formula IVa, the first intermediate comprises a compound according to Formula IVb, the second intermediate comprises a compound according to Formula IVc, and the C-nucleoside 5′ monophosphate comprises a compound according to Formula IVd; 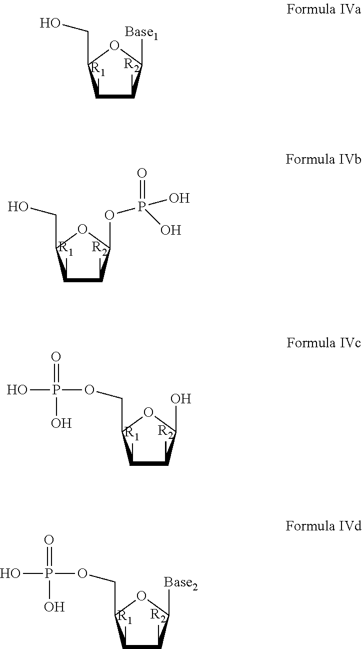
wherein R₁ and R₂ are independently selected from H, OH, OCH₃, F, NH₂, and N₃.

22. The method of claim 21, wherein R₁ and R₂ are each OH.

23. The method of claim 17, wherein the first base and/or second base is selected from uracil, 3-methyluracil, 6-aminouracil, 4-thiouracil, and 2-thiouracil.

24. The method of claim 17, wherein the first base is the same as the second base.

25. The method of claim 24, wherein the second base of step c) is the first base produced as a by-product of the reaction of step a).

26. The method of claim 17, wherein the first base and second base are different.

27. The method of claim 26, wherein the nucleoside 5′-phosphate C-glycosidase is active towards the second base but is inactive towards the first base.

28. The method of claim 26, wherein the first base is selected from cytosine, guanine, thymine, adenine, hypoxanthine, xanthine, 7-methylguanine, 5, 6-dihydrouracil, 5-methylcytosine, and 5-(3-aminoallyl) uracil, and/or the second base is selected from uracil, 3-methyluracil, 6-aminouracil, 4-thiouracil, 2-thiouracil, 2-amino-1H-pyrrole-5-carboxylate, and 1,4-napthoquinone.

29. The method of claim 1, wherein the nucleoside phosphorylase comprises an enzyme selected from purine-nucleoside phosphorylase, pyrimidine nucleoside phosphorylase, thymidine phosphorylase, uridine phosphorylase and guanosine phosphorylase, preferably wherein the nucleoside phosphorylase comprises an enzyme selected from pyrimidine-nucleoside phosphorylase, thymidine phosphorylase and uridine phosphorylase.

30. The method of claim 29, wherein the nucleoside phosphorylase comprises uridine phosphorylase.

31. The method of claim 1, wherein the nucleoside 5′-phosphate C-glycosidase comprises pseudouridylate synthase, optionally wherein the pseudouridylate synthase is selected from YeiN, SdmA, and AlnA.

32. The method of claim 1, wherein the phosphomutase comprises a phosphopentomutase, optionally wherein the phosphpentomutase comprises deoB phosphopentomutase.

33. The method of claim 1, wherein the C-nucleoside-5′-monophosphate obtained in step c) is further treated with alkaline phosphatase.

34. The method of claim 1, wherein the source of phosphate comprises a compound selected from soluble alkaline or alkaline earth metal phosphates, optionally wherein the source of phosphate comprises a compound selected from sodium dihydrogen phosphate, trisodium phosphate, sodium hydrogen phosphate, potassium dihydrogen phosphate, tripotassium phosphate, potassium hydrogen phosphate, a potassium phosphate buffer of about pH 7, and a potassium phosphate buffer of about pH 8.

35. The method of claim 1, wherein steps a), b) and c) are carried out at about 37_° C.

36. The method of claim 1, wherein the C-nucleoside-5′-monophosphate obtained in step c) is purified by one or more methods selected from HPLC, ion-exchange chromatography and reverse phase chromatography.

37. The method of claim 1, wherein the concentration of phosphorylase used in step a) is from 0.02 to 0.6 mg/mL, and/or wherein the concentration of phosphomutase used in step b) is from 0.2 to 0.6 mg/mL, and/or wherein the concentration of the nucleoside 5′-phosphate C-glycosidase used in step c) is from 0.2 to 0.6 mg/mL.

38. The method of claim 1, wherein the enzymes are immobilized on a solid support.
